SEQUENCE LISTING (24 sequences)

SEQ ID NO: 1 - DNA, 56 bp, Artificial Sequence
  Description of Artificial Sequence Synthetic oligonucleotide
gaattcgagctcggtacccggggatccttctagaacgacggcgtcttggagaccct

SEQ ID NO: 2 - DNA, 33 bp, Artificial Sequence
  Description of Artificial Sequence Synthetic oligonucleotide
cgtacgtcggaagactgaccccgaaggccgaca

SEQ ID NO: 3 - DNA, 35 bp, Artificial Sequence
  Description of Artificial Sequence Synthetic oligonucleotide
cttcggggtcagtcttccgacgtacgcgttccagc

SEQ ID NO: 4 - DNA, 56 bp, Artificial Sequence
  Description of Artificial Sequence Synthetic oligonucleotide
caagcttgcatgcctgcaggtcgacttctagaatttcccggaagccagtggtacgc

SEQ ID NO: 5 - DNA, 24 bp, Artificial Sequence
  Description of Artificial Sequence Synthetic oligonucleotide
tctagaagtcgacctgcaggcatg

SEQ ID NO: 6 - DNA, 22 bp, Artificial Sequence
  Description of Artificial Sequence Synthetic oligonucleotide
tctagaaggatccccgggtacc

SEQ ID NO: 7 - DNA, 22 bp, Artificial Sequence
  Description of Artificial Sequence Synthetic oligonucleotide
cttccgacgtacgcgttccagc

SEQ ID NO: 8 - DNA, 23 bp, Artificial Sequence
  Description of Artificial Sequence Synthetic oligonucleotide
ccactgaccccgaaggccgacac

SEQ ID NO: 9 - DNA, 1219 bp, Artificial Sequence
  Description of Artificial Sequence Synthetic polynucleotide
ggtgtgtcggccttcggggtcagtggtacgaacgcccacgtcatcctggaatcggcaccgccgacccagcccgccgacaacgcggtgatcgagcgggcgccggaatggctgcccatggtgatcagtgcacgtacgcagtccgccctgaccgaacacgaaggccggttaagagcgtacctggctgccagcccgggcgtggatatgcgggccgttgcatcgacgttggccatgacacggtcggtgttcgagcaccgcgctgttctgttgggtgacgacaccgtgaccggcaccgcggcgaccgacccgcgtgtcgtcttcgtcttccccggtcagggttcgcagcgggctgggatgggcgaagagttggcagcagcgttcccggtgttcgctcggatccatcagcaggtgtgggacctgctggatgtgccggatctggaggtgaacgagaccgggtacgcgcagccggcgctgttcgccttgcaggtcgctctgttcgggttgttggagtcctggggtgttcgaccggacgcggtggtcggccacagtgtcggagaactggcggccgggtatgtctcgggtctgtggtcgctggaggacgcctgcacgctggtgagcgcaagagcacggctgatgcaggcattacctgctggtggtgtgatggtcgcggtcccggtctccgaagatgaggcgagggcagttttaggtgagggtgtcgagatcgccgccgtgaacggccccagcagcgtggtgctgtccggcgacgaggctgctgtcctgcaggccgctgaggggctggggaagtggacgagattagcaaccagccacgccttccactcggcccggatggagccgatgctggaggagttcaggaccgtcgccgagggcctgacgtaccggaccccgcaggtgtcgatggccgcgggtgatcaggtcaccaccacggagtactgggtgcggcaggttcgtgacacggtccggttcggcgagcaggtggcctcgtacgaggatgcggtgttcgtggagctgggtgcggatcgctcgttggcgagactggtcgacggcgttgcgatgttgcacggcgatcacgaggcccaggcggctgtgtcggcgttggctcacctgtacgtcaacggtgtgacggtggactggccggcattgctgggagatgccccggccacccgggtgctggaccttccgacgtacgcgttccagcaccagc

SEQ ID NO: 10 - DNA, 22 bp, Artificial Sequence
  Description of Artificial Sequence Synthetic oligonucleotide
acgacggcgtcttggagaccct

SEQ ID NO: 11 - DNA, 24 bp, Artificial Sequence
  Description of Artificial Sequence Synthetic oligonucleotide
atttcccggaagccagtggtacgc

SEQ ID NO: 12 - DNA, 16 bp, Artificial Sequence
  Description of Artificial Sequence Synthetic oligonucleotide
acgtcatcctggaatc

SEQ ID NO: 13 - DNA, 16 bp, Artificial Sequence
  Description of Artificial Sequence Synthetic oligonucleotide
acaccgttgacgtaca

SEQ ID NO: 14 - DNA, 40 bp, Artificial Sequence
  Description of Artificial Sequence Synthetic oligonucleotide
accatgattacgcctctagagggtcgacacggcggtctgt

SEQ ID NO: 15 - DNA, 40 bp, Artificial Sequence
  Description of Artificial Sequence Synthetic oligonucleotide
atgacgtgggcgttcgtaccgctgaccccgaaggccgaca

SEQ ID NO: 16 - DNA, 40 bp, Artificial Sequence
  Description of Artificial Sequence Synthetic oligonucleotide
cggccacccgggtgctggaccttccgacgtacgcgttcca

SEQ ID NO: 17 - DNA, 44 bp, Artificial Sequence
  Description of Artificial Sequence Synthetic oligonucleotide
aaacgacggccagttctagaaaggtttcctggaaactcagtgtg

SEQ ID NO: 18 - DNA, 40 bp, Artificial Sequence
  Description of Artificial Sequence Synthetic oligonucleotide
ttccaggaaacctttctagaactggccgtcgttttacaac

SEQ ID NO: 19 - DNA, 40 bp, Artificial Sequence
  Description of Artificial Sequence Synthetic oligonucleotide
cgccgtgtcgaccctctagaggcgtaatcatggtcatagc

SEQ ID NO: 20 - DNA, 44 bp, Artificial Sequence
  Description of Artificial Sequence Synthetic oligonucleotide
tgtcggccttcggggtcagcggtacgaacgcccacgtcatcctg

SEQ ID NO: 21 - DNA, 40 bp, Artificial Sequence
  Description of Artificial Sequence Synthetic oligonucleotide
tggaacgcgtacgtcggaaggtccagcacccgggtggccg

SEQ ID NO: 22 - DNA, 40 bp, Artificial Sequence
  Description of Artificial Sequence Synthetic oligonucleotide
gacgaatctcaaagcagtagaattcccgggtcgactcgag

SEQ ID NO: 23 - DNA, 40 bp, Artificial Sequence
  Description of Artificial Sequence Synthetic oligonucleotide
atggccactcggatcagctcggatccacgcggaaccagat

SEQ ID NO: 24 - DNA, 343 bp, Artificial Sequence
  Description of Artificial Sequence Synthetic polynucleotide
atctggttccgcgtggatccgagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagtagaattcccgggtcgactcgag

(19) United States
(12) Patent Application Publication
(10) Pub. No.: US 2023/0104503 A1
(43) Pub. Date: Apr. 6, 2023

(54) SMALL MOLECULE COMPOUNDS

(71) Applicant: THE REGENTS OF THE UNIVERSITY OF CALIFORNIA, Oakland, CA (US)

(72) Inventors: Edmund GRAZIANI, San Francisco, CA (US); Jay KEASLING, Berkeley, CA (US); Bo PANG, Richmond, CA (US); Tyler BACKMAN, El Cerrito, CA (US); Alan MATHIOWETZ, Waltham, MA (US)

(21) Appl. No.: 17/849,243

(22) Filed: Jun. 24, 2022

Related U.S. Application Data
(60) Provisional application No. 63/215,135, filed on Jun. 25, 2021.

(51) Int. Cl. A61K 31/453

(52) U.S. Cl. CPC A61K 31/453

(57) ABSTRACT

Disclosed herein are pharmaceutical compositions comprising biosynthetic allosteric mTOR inhibitors that can have improved pharmacology and reduced toxicity. Also disclosed herein are methods of treating a condition or disease by administering biosynthetic allosteric mTOR inhibitors.

### CROSS-REFERENCE

This application claims the benefit of U.S. Provisional Application No. 63/215,135, filed on Jun. 25, 2021, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Sep. 23, 2022, is named 59378-701_201_SL.txt and is 7,905 bytes in size.

### BACKGROUND

The emergence of novel RNA viruses as vectors of life-threatening pandemics underlines the urgency for the rapid development of vaccines against these pathogens. Drugs that enhance the ability of the adaptive immune system to respond to viral infections can be useful as adjuvants co-administered with vaccines, thus increasing their effectiveness, particularly in older patients, cancer patients and patients with underlying medical disorders. Productive immunization to viral antigens may hinge on responses from both arms of the adaptive immune system—T and B lymphocytes—and attempts to improve vaccine responses or improve host responses against a viral infection in elderly patients may have to rely on strengthening T-cell immunity.

### SUMMARY

Provided herein are biosynthetic allosteric mTOR inhibitors. In some embodiments, the mTOR inhibitors have improved pharmacology and reduced toxicity. Provided herein certain embodiments are compounds useful as mTOR inhibitors. In some embodiments, disclosed herein is a compound of Formula (I):

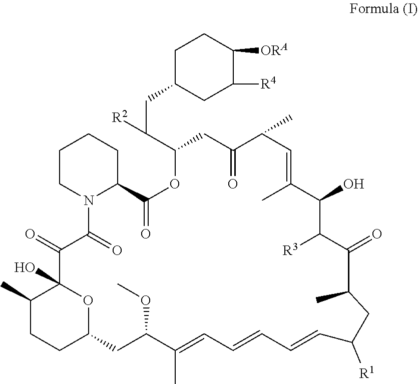

wherein, R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰; R¹ is hydrogen or CH₃; R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰; R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰; R⁴ is —OCH₃ or

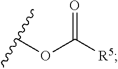

R⁵ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰; each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl; each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a}; R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b}; R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c}; or R²³ and R²⁴ are taken together with the nitrogen atom to which they are attached to form a heterocycloalkyl optionally substituted with one or more R^{1d}; each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl; each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; or R^{c} and R^{d} are taken together with the nitrogen atom to which they are attached to form a heterocycloalkyl optionally substituted with one or more halogen, —OH, —NH₂, or C₁-C₆ alkyl; wherein when R¹ is hydrogen, at least one of (i) R² is not CH₃, (ii) R³ is not —OCH₃, or (iii) R⁴ is not —OCH₃; and wherein when R¹ is CH₃, at least one of (i) R² is not CH₃, (ii) R³ is not hydrogen, —CH₃ or —OCH₃, or (iii) R⁴ is not —OCH₃.

In some embodiments, R⁴ is —OCH₃. In some embodiments, R¹ is hydrogen or CH₃. In some embodiments, R¹ is hydrogen. In some embodiments, R² is CH₃. In some embodiments, R¹ is hydrogen, R² is CH₃, R³ is hydrogen, and R⁴ is —OCH₃. In some embodiments, R¹ is CH₃. In some embodiments, R² is hydrogen or CH₃. In some embodiments, R² is hydrogen. In some embodiments, R¹ is CH₃, R² is hydrogen, R³ is —OCH₃, and R⁴ is —OCH₃. In some embodiments, R¹ is CH₃, R² is hydrogen, R³ is hydrogen, and R⁴ is —OCH₃. In some embodiments, R¹ is hydrogen, R² is CH₃, and R⁴ is —OCH₃. In some embodiments, R^{A} is hydrogen. In some embodiments, R^{A} is —C(═O)R^{B}. When R^{A} is —C(═O)R^{B}, the moiety —O—C(═O)R^{B} comprises an ester group. In some embodiments, the ester group may be cleaved in-vivo. In some embodiments, compounds wherein R^{A} is —C(═O)R^{B} may be a prodrug for a corresponding compound wherein R^{A} is H.

In some embodiments, disclosed herein is a compound of Formula (I-A):

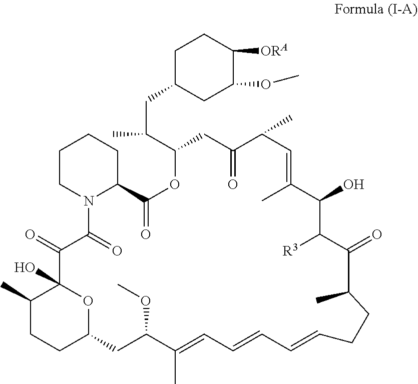

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰; R³ is hydrogen, halogen, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; and
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; or R^{c} and R^{d} are taken together with the nitrogen atom to which they are attached to form a heterocycloalkyl optionally substituted with one or more halogen, —OH, —NH₂, or C₁-C₆ alkyl.

In some embodiments, R^{A} is hydrogen. In some embodiments, R^{A} is —C(═O)R^{B}. When R^{A} is —C(═O)R^{B}, the moiety —O—C(═O)R^{B} comprises an ester group. In some embodiments, R³ is hydrogen.

In some embodiments, disclosed herein is a compound of Formula (I-B):

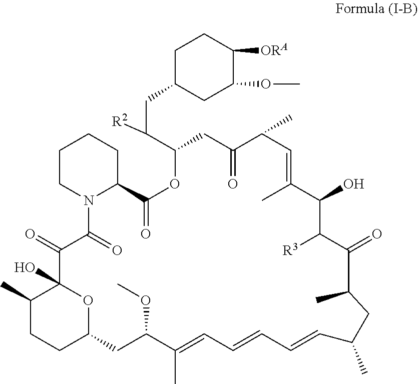

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰; R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₂-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c}; or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; or R^{c} and R^{d} are taken together with the nitrogen atom to which they are attached to form a heterocycloalkyl optionally substituted with one or more halogen, —OH, —NH₂, or C₁-C₆ alkyl; and
wherein when R² is CH₃, R³ is not hydrogen, —CH₃ or —OCH₃.

In some embodiments, R² is hydrogen or CH₃. In some embodiments, R² is hydrogen. In some embodiments, R² is hydrogen and R³ is —OCH₃. In some embodiments, R² is hydrogen and R³ is hydrogen. In some embodiments, R^{A} is hydrogen. In some embodiments, R^{A} is —C(═O)R^{B}. When R^{A} is —C(═O)R^{B}, the moiety —O—C(═O)R^{B} comprises an ester group.

In some embodiments, the compound of Formula (I), or pharmaceutically acceptable salt or solvate thereof, is selected from the group consisting of:

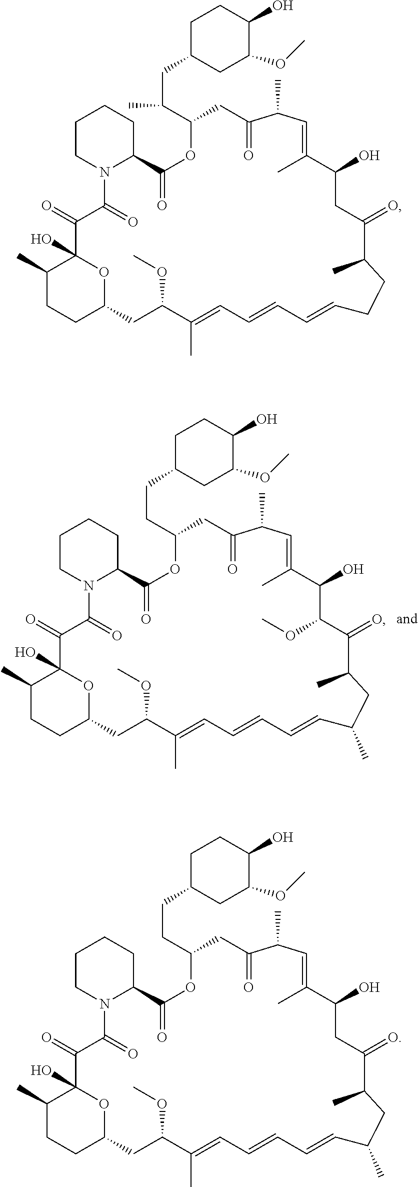

Also provided herein are pharmaceutical compositions comprising a compound with the structure defined by Formula (I), Formula (I-A), or Formula (I-B), and at least one pharmaceutically-acceptable excipient. In some embodiments, the pharmaceutical compositions described herein can be in unit dosage form.

Further provided herein are methods of treating a condition or disease in a subject in need thereof, comprising administering a pharmaceutical composition described herein. Described herein is method of treating a condition or disease in a subject in need thereof, comprising administering a compound described herein or a pharmaceutical composition described herein, thereby treating the condition or disease in the subject. Described herein is use of a compound described herein or a pharmaceutical composition described herein for the treatment of a condition or disease in a subject in need thereof, the use comprising administering to the subject the compound or the pharmaceutical composition, thereby treating the condition or disease in the subject. Described herein is use of a compound described herein or a pharmaceutical composition described herein for the manufacture of a medicament for the treatment of a condition or disease in a subject in need thereof, the use comprising administering to the subject an effective amount of the compound or the pharmaceutical composition, thereby treating the condition or disease in the subject. In some embodiments, administering the compound or the pharmaceutical composition results in inhibiting mTORC1 and/or mTORC2. In some embodiments, administering the pharmaceutical composition further results in promoting immune cell differentiation. In some embodiments, administering the pharmaceutical composition results in a suppression of proliferation of effector T-cells. In some embodiments, administering the pharmaceutical composition further results in differentiation of memory T-cells. In some embodiments, administering the pharmaceutical composition further results in differentiation of regulatory T-cells. In some embodiments, administering the pharmaceutical composition can be in the form of oral administration, rectally administration, parenterally administration, ocular administration, topical administration, intravenous administration, otic administration, inhalation administration, or any combination thereof. In some embodiments, the condition or disease can be a viral infection. In some embodiments, the viral infection is caused by a coronavirus. In some embodiments, the coronavirus is Alphacoronavirus, Betacoronavirus, a Gammacoronavirus, Deltacoronavirus, 229E coronavirus, NL63 coronavirus, OC43 coronavirus, HKUT coronavirus, middle east respiratory syndrome related coronavirus (MERS-CoV), severe acute respiratory syndrome coronavirus (SARS-CoV), severe acute respiratory syndrome coronavirus 2 (SARS-CoV-2), a mutated form of any of these, or any combination thereof. In some embodiments, administering the pharmaceutical composition further comprises co-administration of a vaccine. In some embodiments, co-administration results in improved effectiveness of the vaccine. In some embodiments, administering the pharmaceutical composition is effective to at least partially reduce a viral load of a coronavirus. In some embodiments, the subject has or was previously diagnosed with a general symptom of a coronavirus. In some embodiments, the general symptom comprises a fever, a cough, a shortness of breath, breathing difficulties, or any combination thereof.

Further described herein are kits comprising the pharmaceutical composition described herein. In some embodiments, kits described herein can further comprise instructions for using the pharmaceutical composition. In some embodiments, kits described herein can further comprise a coronavirus vaccine.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure may be obtained by reference to the following detailed description that sets forth illustrative embodiments, in which the principles of the disclosure are utilized, and the accompanying drawings of which:

FIG. 1A shows automatable workflow for in silico design and production via synthetic biology of novel allosteric mTORC1 inhibitors, yielding 5 mg of compounds for testing.

FIG. 1B shows 1,000 low energy conformations of novel macrocycles identified via ClusterCAD screening this disclosure docked to the FRB domain and docking scores were generated both with and without FKBP12 as a binding partner.

FIG. 2A shows measurement of phospho-p70S6K activity via FACS analysis upon activated mouse T-cells (whole splenocyte from OT1 B6 mice) stimulated by soluble anti-CD3+ (3 ug/mL), anti-CD28 (2 ug/mL), and IgG (1.5 ug/mL) following overnight treatment with compounds RAP23, RAP23/27, RAP35, RAP35/27A, and RAP35/27B.

FIG. 2B shows graphs that displays the effect on proliferation of CD8+ cells isolated from p14 transgenic mice labeled with CellTrace™ Violet (CTV) dye, activated for 72 hours with anti-CD3+ (5 ug/mL) and anti-CD28 (2 ug/mL) with 3 concentrations (10 nM, 100 nM, and 1 uM) of RAP23 and rapamycin (control).

FIG. 2C shows FACS analysis of naïve CD4+ mouse T-cells stimulated with soluble anti-CD3+ (3 ug/mL) and anti-CD28 (2 ug/mL) in the presence of 1000 nM RAP23 and rapamycin for CD4+CD25+FOXP3+ relative to untreated DMSO control.

FIG. 2D shows FACS analysis of naïve CD4+ mouse T-cells stimulated with soluble anti-CD3+ (3 ug/mL) and anti-CD28 (2 ug/mL) in the presence of increasing concentrations (1 nM to 1000 nM) RAP23 and rapamycin for CD8+CD6L2+ relative to untreated DMSO control.

FIG. 3 shows the computational pipeline for gene cluster modifications.

FIG. 4A shows the mechanism of S. rapamycinicus genomic DNA manipulation via homologous recombination. LA, left arm. RA, right arm. MR, modified region.

FIG. 4B shows the workflow of S. rapamycinicus genomic DNA manipulation via homologous recombination.

FIG. 5 shows the swap of acyltransferase domain (AT) in module 7 of the rapamycin polyketide synthase (PKS) assembly line to produce 23-desmethylrapamycin analogs. The circles (top) indicate desired and resulting changes (bottom).

FIG. 6 shows the swap of the acyltransferase domain (AT) in module 1 of the rapamycin polyketide synthase (PKS) assembly line to produce 35-desmethylrapamycin analogs. The circles (top) indicate desired and resulting changes (bottom).

FIG. 7 shows the proposed swap of the AT in module 14 of the rapamycin PKS to produce 9-methylrapamycin.

FIG. 8A shows chromatogram graphs of HPLC-DAD analysis of the fermentation broth of S. rapamycinicus RAPA016 of absorbance at 278 nm wavelength.

FIG. 8B shows graphs of UV absorption spectra of rapamycin, 23-desmethylrapamycin (RAP23), and 23-desmethyl-27-demethoxyrapamycin (RAP23/27).

FIG. 9A shows chromatogram graphs of HPLC-DAD analysis of the fermentation broth of S. rapamycinicus RAPA060 of absorbance at 278 nm wavelength.

FIG. 9B shows graphs of UV absorption spectra of rapamycin, 35-desmethylrapamycin (RAP35), and 35-desmethyl-27-demethoxyrapamycin (RAP35/27).

FIG. 10 shows SPR single-cycle kinetics of FKBP-rapamycin analog-FRB ternary complex formation. The experimental sensorgrams of titration of FRB to immobilized FKBP-rapamycin analog complex was fitted to 1:1 binding model.

FIG. 11 shows the percentage of p-S6^{hi} cells in activated T cells treated with rapamycin or rapamycin analog at different time points after activation.

FIG. 12 shows CD8 CTV-labelled proliferation with mTOR inhibitors at 48 hours (top 3 graphs) and 72 hours (bottom 3 graphs).

FIG. 13 shows Treg generation in the presence of IL-2 and TGFb, with treatment of rapamycin analogs or rapamycin. FOXP3 was gated as the indicator.

FIG. 14 shows the rapamycin analogs increase CD62L+ Tregs.

### DETAILED DESCRIPTION

While various embodiments of the invention have been shown and described herein, it may be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Terminology

Unless specifically stated or obvious from context, as used herein, the term “about” in reference to a number or range of numbers is understood to mean the stated number and numbers +10% thereof, or 10% below the lower listed limit and 10% above the higher listed limit for the values listed for a range.

The singular forms “a”, “an”, and “the” include plural referents unless the context clearly dictates otherwise. Thus, for example, reference to “the surfactant” includes reference to one or more specific surfactants, reference to “an antioxidant” includes reference to one or more of such additives.

The term “subject” as used herein refers to a mammal (e.g., a human, mouse, rat, guinea pig, dog, cat, horse, cow, pig, or non-human primate, such as a monkey, chimpanzee or baboon).

“Effective amount,” “sufficient amount,” and “amount sufficient for” may be used interchangeably and refer to an amount of a substance that is sufficient to achieve an intended purpose or objective.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

A “therapeutically effective amount” when used in connection with a pharmaceutical composition described herein is an amount of one or more pharmaceutically active agent(s) sufficient to produce a therapeutic result in a subject in need thereof. An “amount” of one or more components in the pharmaceutical composition refers to an amount per unit dose.

The term “pharmaceutically-acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically-acceptable” can refer a material, such as a carrier or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

The term “derivative” as used herein indicates a chemical or biological substance that is related structurally to a second substance and derivable from the second substance through a modification of the second substance. In particular, if a first compound is a derivative of a second compound and the second compound is associated with a chemical and/or biological activity, the first compound differs from the second compound for at least one structural feature, while retaining (at least to a certain extent) the chemical and/or biological activity of the second compound and at least one structural feature (e.g. a sequence, a fragment, a functional group and others) associated thereto.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In various instances, “may” refers to optional alternatives to be used in the alternative or in addition to other specified components.

“Alkyl” refers to a straight or branched hydrocarbon chain radical consisting solely of carbon and hydrogen atoms, which may optionally be unsaturated with one or more double or triple bonds, and preferably having from one to fifteen carbon atoms (i.e., C₁-C₁₅ alkyl). In certain embodiments, an alkyl comprises one to six carbon atoms (i.e., C₁-C₆ alkyl). In other embodiments, an alkyl comprises one to three carbon atoms (i.e., C₁-C₃ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless otherwise specified, the term “alkyl” and its equivalents encompass linear, branched, and/or cyclic alkyl groups. In some instances, an “alkyl” comprises both cyclic and acyclic (linear and/or branched) alkyl components. When an alkyl group is described as “linear,” the referenced alkyl group is not substituted with additional alkyl groups and is unbranched. When an alkyl group is described as “saturated,” the referenced alkyl group does not contain any double or triple carbon-carbon bonds (e.g. alkene or alkyne).

“Alkylene” or “alkylene chain” refers to a divalent alkyl group, which may be saturated or unsaturated with one or more double or triple bonds.

“Aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C_{x-y}alkyl” refers to saturated or unsaturated hydrocarbon groups, including straight-chain alkyl and branched-chain alkyl groups that contain from x to y carbons in the chain. The terms “C_{x-y}alkenyl” and “C_{x-y}alkynyl” refer to unsaturated aliphatic groups analogous in length and possible substitution to the alkyls described above, but that contain at least one double or triple bond respectively.

“Cycloalkyl” refers to a saturated ring in which each atom of the ring is carbon. Cycloalkyl may include monocyclic and polycyclic rings such as 3- to 10-membered monocyclic rings, 6- to 12-membered fused bicyclic rings, 6- to 12-membered spirocyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like.

“Halo” or, alternatively, “halogen” or “halide,” means fluoro, chloro, bromo or iodo. In some embodiments, halo is fluoro, chloro, or bromo.

“Haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally substituted as described herein.

“Heteroalkyl” refers to an alkyl group wherein one or more of the carbons of the alkyl group is replaced with a heteroatom. Exemplary heteroatoms include N, O, Si, P, B, and S atoms, preferably N, O and S. Note that valency of heteroatoms may not be identical to that of a carbon atom, so, for example, a methylene (CH₂) of an alkyl may be replaced with an NH group, S group, O group, or the like in a heteroalkyl.

“Heteroalkylene” refers to an alkylene group wherein one or more of the carbons of the alkylene group is replaced with a heteroatom. Exemplary heteroatoms include N, O, Si, P, B, and S atoms, preferably N, O and S.

“Heterocycloalkyl” refers to a saturated or unsaturated (e.g., non-aromatic) ring with carbon atoms and at least one heteroatom (e.g., a cycloalkyl wherein one or more of the carbon groups is substituted with a heteroatom). Exemplary heteroatoms include N, O, Si, P, B, and S atoms. Heterocycloalkyl may include monocyclic and polycyclic rings such as 3- to 10-membered monocyclic rings, 6- to 12-membered fused bicyclic rings, 6- to 12-membered spirocyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heteroaryl” refers to an aromatic ring comprising carbon atoms and one or more heteroatoms. Exemplary heteroatoms include N, O, Si, P, B, and S atoms. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl).

The term “substituted” refers to moieties having substituents replacing a hydrogen on one or more carbons or heteroatoms of the structure. It may be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. In embodiments where it is unspecified whether a group is substituted or unsubstituted, it is intended that the group is unsubstituted.

Substituents may include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{e} is a straight or branched alkylene, alkenylene or alkynylene chain.

Compounds of the present invention also include crystalline and amorphous forms of those compounds, pharmaceutically-acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

### MTOR Inhibitors

Mechanistic target of rapamycin (mTOR) is a highly conserved member of the phosphatidylinositol 3-kinase family that exists in two protein complexes, mTORC1 and mTORC2. Inhibition of mTORC1 signaling by the microbial natural product, rapamycin (sirolimus) and its cytosolic chaperone immunophilin, FK-506 binding protein 12 (FKBP12), suppresses proliferation of effector T-cells and may induce differentiation of memory and regulatory T-cells (T_{reg}) in humans. Regulatory T-cells are a subset of anti-inflammatory T cells that dampen an immune response through secretion of immunomodulatory cytokines (e.g., IL-10, TGFβ), checkpoint inhibitor interaction (e.g., CTLA-4, LAG-3) and competition for essential metabolites and cytokines. Without wishing to be bound to any theory, for a therapeutic to maximize efficacy within an autoimmune disease setting, it would need to reduce conventional T cell proliferation and activation whilst preserving or enhancing the generation or phenotype of T_{reg} cells. mTORC1 inhibition may promote immune cell differentiation that can lead to more robust and effective responses to vaccines. The immune-enhancing effects of rapamycin have been shown for CD8+ T cells.

Inhibitors of mTORC1/C2 may significantly reduce infections in the elderly when co-administered with a seasonal influenza vaccine. The combination of an orthotopic inhibitor at the ATP-binding site of mTORC1 (dactolisib, a pan-phosphoinositide 3-kinase (PI3K)/mTOR inhibitor) and an allosteric mTORC1 inhibitor (everolimus) administered for 6 weeks prior to immunization in a Phase II clinical study showed significantly increased antibody titers to influenza virus vaccine strains, and reduced the aforementioned incidence of respiratory infections for a year. A Phase 3 clinical study focusing on determining if using dactolisib alone prevents illness associated with respiratory tract infections in people ≥65 years of age. However, the study was discontinued and the rationale used for testing dactolisib alone (without everolimus) in this study is unclear, which left open the possibility that the positive results observed in the Phase II study were attributable, wholly or in part, to everolimus. This observation outlined a path to discover a single agent that can selectively modulate the role of mTOR in both innate and adaptive responses in T-cells that would have significant therapeutic potential for protecting the population from viral respiratory infections. Interestingly, a SARS-CoV-2-human protein-protein interaction map revealed direct viral-human interactions with proteins regulated by the mTORC1 pathway, such as LARP1, and FKBP7, which interact with the viral N and Orf8 proteins, respectively, suggesting the added possibility of direct mTORC1 inhibition affecting viral replication.

The rapidity with which SARs-CoV2 vaccines are being developed is quite impressive, with at multiple clinical trials already up and concurrently running. This, however, makes even more urgent the need to develop therapeutics to ensure that these vaccines are effective in the most vulnerable populations. Therefore, there is a need to design and screen for compounds that are partial allosteric antagonists of mTORC1 signaling such that downstream events that control proliferation are ablated, while effects on differentiation to regulatory and memory phenotypes are preserved. In this way, a single agent can combine sufficient but partial mTORC1 inhibition to enhance innate antiviral immunity while at the same time possessing an optimized magnitude and duration of inhibition that preserves and potentially improves the enhancement of adaptive immunity.

### Rapamycin and Derivatives

Previous medicinal chemistry work on rapamycin was focused on designing compounds for use in treating cancer, and thus the observation that modifications to the macrocyclic ring of rapamycin led to reduced antiproliferative activity of tumor cells led to the prioritization of compounds that retained full binding to mTORC1. Hence, the four rapamycin analogs that are currently FDA approved are essentially identical, as all are modified with solubilizing groups at the C-42 hydroxyl position.

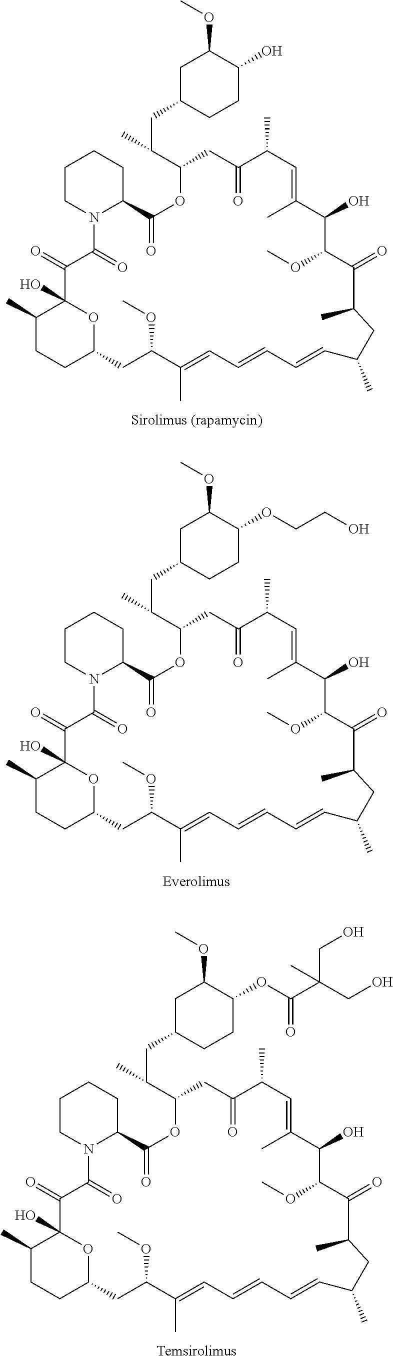

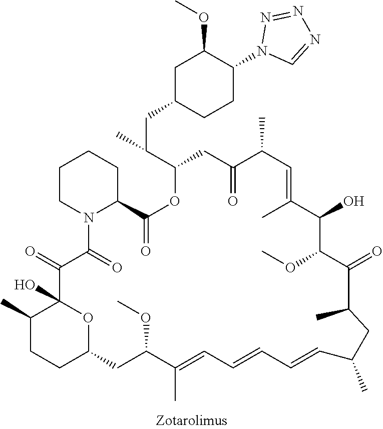 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2CC[C@@H](n3cnnn3)[C@H](OC)C2)CC(=O)[C@H](C)/C=C(\C)[C@@H](O)[C@@H](OC)C(=O)[C@H](C)C[C@H](C)/C=C/C=C/C=C/1C]

### Optimization of Analogs

### Computational Design Optimization

In some embodiments, the compounds described herein consider the structural features that can be important for binding to FKBP12 and combined with additional variables to alter the terminal half-life in vivo. As partitioning into the red blood cells is thought to be driven by binding to immunophilins such as FKBPs, this may spare the compounds described herein from first pass metabolism and lead to the long terminal half-life (24-48 hours) observed in humans with rapamycin.

In contrast to previous methods for optimizing rapamycin analogs, the compounds described herein were designed by employing a structure-based drug design approach using a rapamycin engineering simulator to predict the effect of specific structural changes to the natural product on their binding to both the FRB domain of mTOR and/or to FKBP12 via computational docking. The computational tool described herein was used for prioritization of potential engineering modifications to the rapamycin biosynthetic gene cluster, based on predicted engineering accessibility and chemical properties of the resulting chemical products, and simulating biosynthesis of rapamycin.

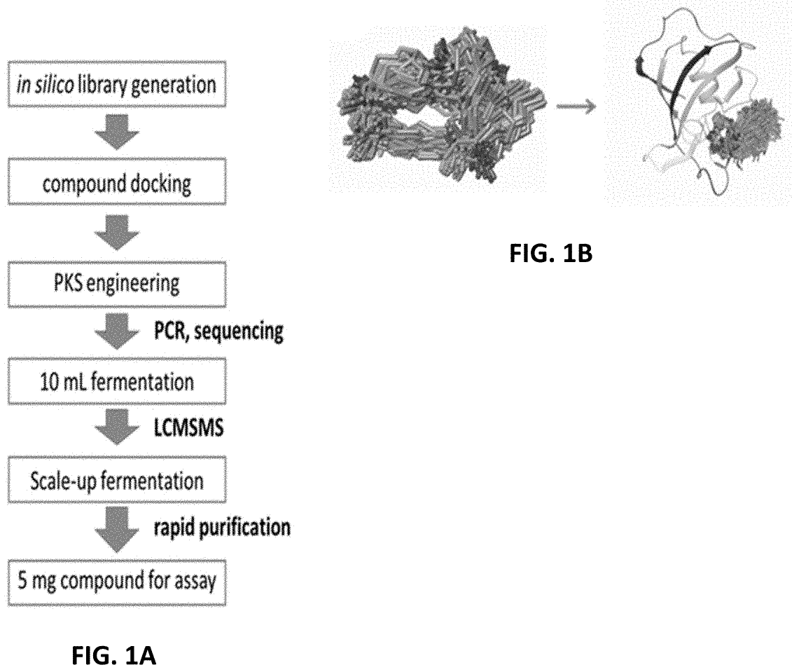

The rapamycin engineering simulator described herein is a computational tool designed to simulate the biosynthesis of and engineering modifications to rapamycin. It is a Python language Jupyter notebook that allows for interactive use, and visualization of chemical structures. It was written as an extension to the open source PKS engineering software ClusterCAD. Every enzymatic step in the biosynthesis of rapamycin was represented as a software object, which can computationally simulate the chemical reaction performed by the real enzyme it represents using Reaction SMARTS operators, including correct stereochemistry. All PKS, NRPS, and post-PKS/NPRS tailoring enzymes were included, and functional. This computational tool described herein rapidly generated new libraries of engineering accessible rapamycin analogues by deleting or swapping out the enzyme objects for others, in a manner analogous to domain swapping in a real PKS/NRPS enzyme (FIG. 1). The libraries of new chemical structures that could be made by common engineering changes to PKS gene clusters: acyltransferase (AT) domain swaps, gene cluster truncations, and post-PKS tailoring enzyme knockouts. FIG. 1A shows an overall scheme from computational design to biological assay testing of engineered compounds. For each structure, ˜1000 low-energy conformations were generated using Prime macrocycle search, each structure was then docked against crystal structures of the human FKBP12-rapamycin-FRB ternary complex with rapamycin deleted, as well as mTOR alone (FKBP12 and rapamycin deleted) using flexible docking Glide SP with Prime Macrocycle sampling (20-50 hr/cpd) (FIG. 1B). A mTOR/ternary docking score ratio was calculated using the docking scores and a scoring system. The docking score was used for predicting the relative binding of the structures described herein to FKBP12 vs mTOR. These docking results were used to make informed decisions about which target chemical structures to prioritize, and corresponding rapamycin gene cluster modifications to perform. The computational technique can further employ its docking results for calculating cryo-electron micrograph (cryo-EM) structures of mTORC1 to identify binding events at the FRB domain of mTOR with various substrates Analysis of data from cryo-EM can provide insight on key substrates which may allow for a focused effort to improve the efficacy and safety of drugs in this space.

In some embodiments, the compounds described herein can be synthesized using synthetic biology techniques to engineer the rapamycin polyketide synthase in Streptomyces rapamycinicus for producing specific alterations to the structure of products generated through exchange acyltransferases that specify substituents at the α-carbons of the Claisen-type products of PKS assembly and accompanying cytochrome P-450s.

### Compounds

The present disclosure provides compounds and salts, and formulations thereof, for use in treating various diseases.

In some embodiments, disclosed herein is a compound, or a pharmaceutically-acceptable salt or solvate thereof, having a structure represented by a structure of Formula (I):

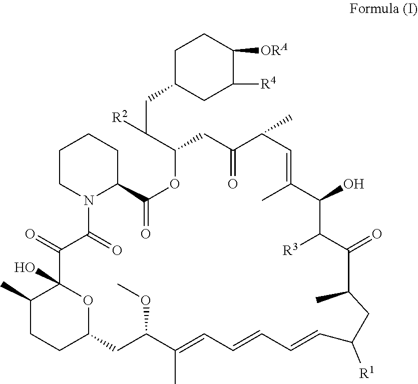

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R¹ is hydrogen or CH₃;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰
R⁴ is —OCH₃ or

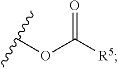

R⁵ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl;
wherein when R¹ is hydrogen, at least one of (i) R² is not CH₃, (ii) R³ is not —OCH₃, or (iii) R⁴ is not —OCH₃;
and
wherein when R¹ is CH₃, at least one of (i) R² is not CH₃, (ii) R³ is not hydrogen, —CH₃ or —OCH₃, or (iii) R⁴ is not —OCH₃.

In some embodiments, disclosed herein is a compound, or a pharmaceutically-acceptable salt or solvate thereof, having a structure represented by a structure of Formula (I′):

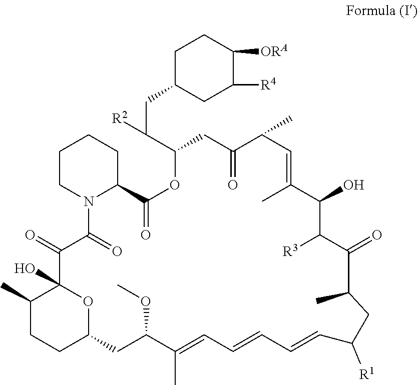

wherein,
R^{A} is hydrogen or —C(═O)R^{B}, wherein R^{B} is alkyl, aryl, heterocycloalkyl, or heteroaryl; wherein the alkyl, aryl, heterocycloalkyl, or heteroaryl are each independently optionally substituted;
R¹ is hydrogen or CH₃;
R² is hydrogen, halogen, —CN, alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R³ is hydrogen, halogen, —OCH₃, —CN, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
R⁴ is —OCH₃ or

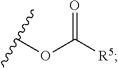

R⁵ is hydrogen, halogen, —CN, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl, wherein the alkoxy, thioalkoxy, nitro, amino, amido, sulfoxide, sulfonyl, sulfonylamino, aminosulfonyl, acyl, oxalyl, carbamyl, carbonyl, ureido, ester, alkyl, aryl, heterocycloalkyl, or heteroaryl are each independently optionally substituted;
wherein when R¹ is hydrogen, at least one of (i) R² is not CH₃, (ii) R³ is not —OCH₃, or (iii) R⁴ is not —OCH₃; and
wherein when R¹ is CH₃, at least one of (i) R² is not CH₃, (ii) R³ is not hydrogen, —CH₃ or —OCH₃, or (iii) R⁴ is not —OCH₃.

In some embodiments of Formula (I) or (I′), R^{A} is hydrogen. In some embodiments of Formula (I) or (I′), R^{A} is —C(═O)R^{B}. When R^{A} is —C(═O)R^{B}, the moiety —O—C(═O)R^{B} comprises an ester group. In some embodiments, the ester group may be cleaved in-vivo. In some embodiments, compounds wherein R^{A} is —C(═O)R^{B} may be a prodrug for a corresponding compound wherein R^{A} is H.

In some embodiments of Formula (I) or (I′), R¹ is alkyl which is optionally substituted.

In some embodiments of Formula (I) or (I′), R² is alkyl which is optionally substituted. In some embodiments of Formula (I) or (I′), R³ is alkoxy, which may be optionally substituted. In some embodiments of Formula (I) or (I′), R⁴ is alkoxy, which may be optionally substituted.

In some embodiments of Formula (I) or (I′), R⁴ is —OCH₃. In some embodiments of Formula (I) or (I′), R¹ is hydrogen or CH₃. In some embodiments of Formula (I) or (I′), R¹ is hydrogen. In some embodiments of Formula (I) or (I′), R² is CH₃. In some embodiments of Formula (I) or (I′), R¹ is hydrogen; R² is CH₃; R³ is hydrogen; and R⁴ is —OCH₃.

In some embodiments of Formula (I) or (I′), R¹ is CH₃. In some embodiments of Formula (I) or (I′), R² is hydrogen or CH₃. In some embodiments of Formula (I) or (I′), R² is hydrogen. In some embodiments of Formula (I) or (I′), R¹ is CH₃; R² is hydrogen; R³ is —OCH₃; and R⁴ is —OCH₃. In some embodiments of Formula (I) or (I′), R¹ is CH₃; R² is hydrogen; R³ is hydrogen; and R⁴ is —OCH₃.

In some embodiments of Formula (I) or (I′), R¹ is hydrogen, R² is CH₃, and R⁴ is —OCH₃.

In some embodiments, disclosed herein is a compound, or a pharmaceutically-acceptable salt or solvate thereof, having a structure represented by a structure of Formula (I-A):

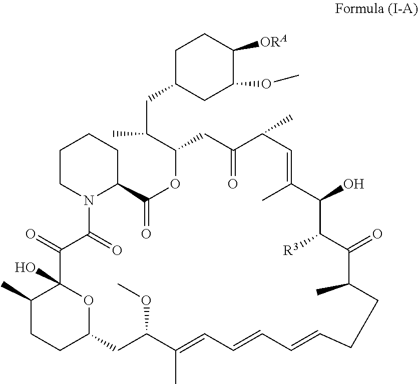

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl; and
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl.

In some embodiments of Formula (I-A), R³ is hydrogen. In some embodiments of Formula (I-A), R^{A} is hydrogen. In some embodiments of Formula (I-A), R^{A} is —C(═O)R^{B}. When R^{A} is —C(═O)R^{B}, the moiety —O—C(═O)R^{B} comprises an ester group.

In some embodiments, disclosed herein is a compound, or a pharmaceutically-acceptable salt or solvate thereof, having a structure represented by a structure of Formula (I-B):

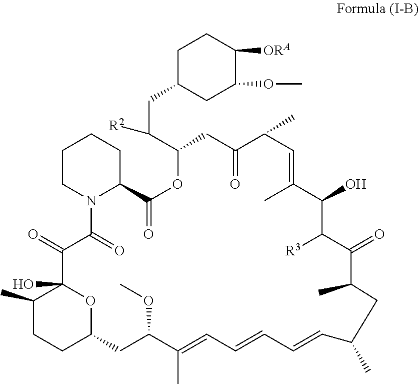

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl;
and
wherein when R² is CH₃, R³ is not hydrogen, —CH₃ or —OCH₃.

In some embodiments of Formula (I-B), R² is alkyl which is optionally substituted. In some embodiments of Formula (I), R³ is alkoxy, which may be optionally substituted.

In some embodiments of Formula (I-B), R^{A} is hydrogen. In some embodiments of Formula (I-B), R^{A} is —C(═O)R^{B}. When R^{A} is —C(═O)R^{B}, the substituent —O—C(═O)R^{B} comprises an ester group. In some embodiments, compounds wherein R^{A} is —C(═O)R^{B} may be a prodrug for a corresponding compound wherein R^{A} is H.

In some embodiments of Formula (I-B), R² is hydrogen or CH₃. In some embodiments of Formula (I-B), R² is hydrogen. In some embodiments of Formula (I-B), R³ is hydrogen. In some embodiments of Formula (I-B), R² is hydrogen and R³ is —OCH₃. In some embodiments of Formula (I-B), R² is hydrogen and R³ is hydrogen.

In some embodiments, the compound of Formula (I) is

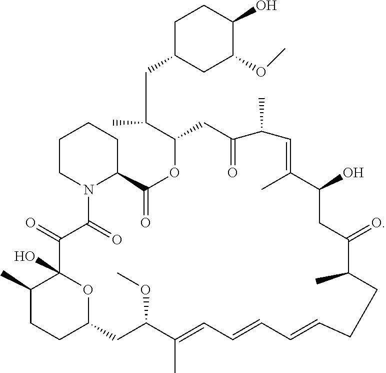 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)CC(=O)[C@H](C)CC/C=C/C=C/C=C/1C]

In some embodiments, the compound of Formula (I) is

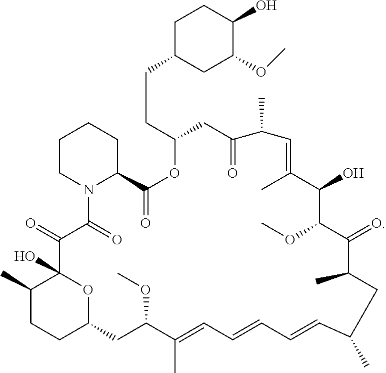 [SMILES: CO[C@H]1C[C@@H]2CC[C@@H](C)[C@@](O)(O2)C(=O)C(=O)N2CCCC[C@H]2C(=O)O[C@H](CC[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the compound of Formula (I) is

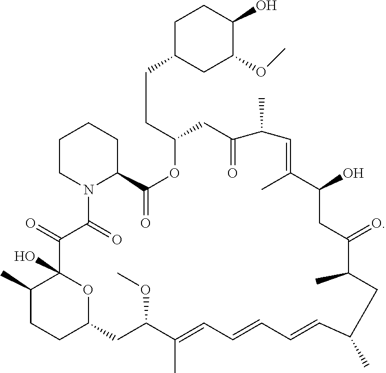 [SMILES: CO[C@H]1C[C@@H]2CC[C@@H](C)[C@@](O)(O2)C(=O)C(=O)N2CCCC[C@H]2C(=O)O[C@H](CC[C@@H]2CC[C@@H](O)[C@H](OC)C2)CC(=O)[C@H](C)/C=C(\C)[C@@H](O)CC(=O)[C@H](C)C[C@H](C)/C=C/C=C/C=C/1C]

In some embodiments, the compounds of Formula (I), (I-A), and (I-B) has improved binding to mTOR (e.g., mTORC1, mTORC2). In some embodiments, the compounds of Formula (I), (I-A), and (I-B) has improved binding to immunophilins (e.g., FKBP12). In some embodiments, the compounds of Formula (I), (I-A), and (I-B) has improved terminal half-life in vivo and may evade first pass metabolism. In some embodiments, the compounds of Formula (I), (I-A), and (I-B) have improved solubility.

### Pharmaceutical Compositions

Pharmaceutically-Acceptable Salts

Provided herein are pharmaceutically-acceptable salts of the compounds described herein. As used herein, a pharmaceutically-acceptable salt includes, but is not limited to, acid addition salts or basic addition salts. Pharmaceutically-acceptable salts include, but are not limited to, alkali metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Examples of pharmaceutically-acceptable salts include, but are not limited to, bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorohydrate, and sulfate pentahydrate. Other representative pharmaceutically-acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically-acceptable salt.

Excipients

In some embodiments, a pharmaceutical composition can comprise an excipient. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical composition.

In some embodiments an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁-C₁₈ fatty acid alcohol, polyethylene glycol, polyols, saccharides, oligosaccharides, and combinations thereof.

The binders that can be used in a pharmaceutical composition can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments a pharmaceutical composition can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical composition can be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical composition can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition can comprise a disintegrant as an excipient. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like.

In some instances, a pharmaceutical composition can comprise a coloring agent. Non-limiting examples of suitable color agents can include food, drug and cosmetic colors (FD&C), drug and cosmetic colors (D&C), and external drug and cosmetic colors (Ext. D&C). A coloring agent can be used as dyes or their corresponding lakes.

In some instances, a pharmaceutical composition can comprise anti-adherents (anti-sticking agents, glidants, flow promoters, lubricants) (e.g., talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate) anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4hydroxymethyl-2,6-di-tert-butyl phenol, tocopherol, etc.), binders (adhesives), i.e., agents that impart cohesive properties to powdered materials through particle-particle bonding (e.g., matrix binders (dry starch, dry sugars), film binders (PVP, starch paste, celluloses, bentonite, sucrose)), chemical binders (e.g., polymeric cellulose derivatives, such as carboxy methyl cellulose, HPC, HPMC, etc., sugar syrups, corn syrup, water soluble polysaccharides (e.g., acacia, tragacanth, guar, alginates, etc), gelatin, gelatin hydrolysate, agar, sucrose, dextrose, non-cellulosic binders (e.g., PVP, PEG, vinyl pyrrolidone copolymers, pregelatinized starch, sorbitol, glucose, etc.), bufferants, where the acid is a pharmaceutically-acceptable acid, (e.g., hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, etc) and where the base is a pharmaceutically-acceptable base (e.g., an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine), or a pharmaceutically-acceptable salt of acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, parabromophenylsulfonic acid, propionic acid, p-toluenesulfonic acid, salicylic acid, stearic acid, succinic acid, tannic acid, tartaric acid, thioglycolic acid, toluenesulfonic acid, and uric acid, chelating agents (e.g., EDTA and EDTA salts), coagulants (e.g., alginates) colorants or opaquants (e.g., titanium dioxide, food dyes, lakes, natural vegetable colorants, iron oxides, silicates, sulfates, magnesium hydroxide and aluminum hydroxide), coolants (e.g. halogenated hydrocarbons (e.g., trichloroethane, trichloroethylene, dichloromethane, fluorotrichloromethane), diethylether and liquid nitrogen) cryoprotectants (e.g., trehelose, phosphates, citric acid, tartaric acid, gelatin, dextran, mannitol, etc.), diluents or fillers (e.g., lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose disintegrants or super disintegrants (e.g., croscarmellose sodium, starch, starch derivatives, clays, gums, cellulose, cellulose derivatives, alginates, crosslinked polyvinylpyrrolidone, sodium starch glycolate and microcrystalline cellulose), hydrogen bonding agents (e.g., magnesium oxide), flavorants or desensitizers (e.g., spray-dried flavors, essential oils and ethyl vanillin), ion-exchange resins (e.g., styrene/divinyl benzene copolymers, and quaternary ammonium compounds), plasticizers (e.g., polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate), preservatives (e.g., ascorbic acid, boric acid, sorbic acid, benzoic acid, and salts thereof, parabens, phenols, benzyl alcohol, and quaternary ammonium compounds), solvents (e.g., alcohols, ketones, esters, chlorinated hydrocarbons and water) sweeteners, including natural sweeteners (e.g., maltose, sucrose, glucose, sorbitol, glycerin and dextrins), and artificial sweeteners (e.g., aspartame, saccharine and saccharine salts) and thickeners (viscosity modifiers, thickening agents), (e.g., sugars, polyvinylpyrrolidone, cellulosics, polymers and alginates).

In some instances, a pharmaceutical composition can comprise proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, camuaba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics, (e.g., dicalcium phosphate, hydroxyapitite, tricalcium phosphate, talc and titania), polyols (e.g., mannitol, xylitol and sorbitol polyethylene glycol esters) and polymers (e.g., alginates, poly(lactide coglycolide), gelatin, crosslinked gelatin and agar-agar).

In some instances, a pharmaceutical composition can comprise adsorbents. Many adsorbents are solid, porous or super porous adsorption materials. They comprise numerous micro- or nano-pores within their structures, resulting in very large surface areas, for example, greater than 500 m²/g. Exemplary absorbents include, without limitation, silica, active carbon, magnesium aluminum silicate, and diatomite.

In some embodiments, a compound described herein can be present in the form of a prodrug. The term “prodrug” as used herein, can refer to a drug precursor that, following administration to a subject and subsequent absorption, can be converted to an active, or a more active species via some process, such as conversion by a metabolic pathway. Thus, the term can encompass a derivative, which, upon administration to a recipient, can be capable of providing, either directly or indirectly, a compound, salt or a metabolite thereof. Some prodrugs can have a chemical group present on a prodrug that renders it less active and/or confers solubility or some other property to the drug. Once the chemical group has been cleaved and/or modified from the prodrug the active drug can be generated. Prodrugs can increase the bioavailability of a compound described herein when administered to a subject (e.g. by allowing an administered compound described herein to be more readily absorbed) or which enhance delivery of the compound described herein to a biological compartment (e.g. the brain or lymphatic system).

In some embodiments, prodrugs include compounds where ester groups are bonded to any group that, when administered to a mammalian subject, cleaves to form a free hydroxyl. In some embodiments, compounds of the present disclosure are prodrugs comprising an ester group, wherein the ester group may be cleaved in-vivo to generate a compound having a hydroxyl group at the corresponding position.

Administration

In some embodiments, a pharmaceutical formulation disclosed herein can be formulated into a variety of forms and administered by a number of different means. In some cases, a pharmaceutical formulation can be biodegradable. A pharmaceutical formulation can be administered orally, rectally, parenterally, ocular administration, topically, intravenously, otic administration, by inhalation administration, intranasally, in formulations containing conventionally acceptable carriers, adjuvants, and vehicles as desired. The term “parenteral” as used herein can include subcutaneous, intravenous, intramuscular, or intrasternal injection and infusion techniques. Administration can include injection or infusion, including intra-arterial, intracardiac, intracerebroventricular, intradermal, intraduodenal, intramedullary, intramuscular, intraosseous, intraperitoneal, intrathecal, intratracheal, intravascular, intravenous, intravitreal, epidural and subcutaneous, inhalational, transdermal, transmucosal, sublingual, buccal and topical (including epicutaneous, dermal, enema, eye drops, ear drops, intranasal, vaginal) administration. In some exemplary embodiments, a route of administration can be via an injection such as an intramuscular, intravenous, subcutaneous, intratracheal, or intraperitoneal injection. In some cases, an administering is a systemic administering. A systemic administering may be, for example, a parenteral injection at a site that allows for circulation.

Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion.

Tablets, pills, and the like can be compressed, multiply compressed, multiply layered, and/or coated. A coating can be single or multiple.

Liquid formulations can include a syrup (for example, an oral formulation), an intravenous formulation, an intranasal formulation, an ocular formulation (e.g., for treating an eye infection), an otic formulation (e.g., for treating an ear infection), an ointment, a cream, an aerosol, and the like. In some cases, a liquid formulation can comprise a gel microsphere, or caulking hydrogel. In some instances, a combination of various formulations can be administered. In some embodiments, a tablet, pill, and the like can be formulated for an extended release profile.

Drops, such as eye drops or nose drops, may be formulated with one or more of a pharmaceutical composition in an aqueous or non-aqueous base also comprising one or more dispersing agents, solubilizing agents or suspending agents. Liquid sprays can be pumped or are conveniently delivered from pressurized packs. Drops can be delivered via a simple eye dropper-capped bottle, via a plastic bottle adapted to deliver liquid contents drop-wise, or via a specially shaped closure.

In some instances, a pharmaceutical composition described herein can be administered in a composition for topical administration. For topical administration, an active agent may be formulated as is known in the art for direct application to a target area. Forms chiefly conditioned for topical application can take the form, for example, of creams, milks, gels, powders, dispersion or microemulsions, lotions thickened to a greater or lesser extent, impregnated pads, ointments or sticks, aerosol formulations (e.g. sprays or foams), hydrogel, soaps, detergents, lotions or cakes of soap. Other conventional forms for this purpose include wound dressings, coated bandages or other polymer coverings, ointments, creams, lotions, pastes, jellies, sprays, and aerosols. Thus, a pharmaceutical composition disclosed herein can be delivered via patches or bandages for dermal administration.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. In some embodiments, a pharmaceutical composition can comprise the compound described herein and at least one excipient.

In some embodiments, the pharmaceutical composition described herein is in the form of a unit dose. In some embodiments, the pharmaceutical composition can be co-administered with a vaccine. In some embodiments, the pharmaceutical composition can be an adjuvant to a vaccine. In some embodiments, the pharmaceutical composition increases the efficacy and improve the effectiveness of a vaccine. In some embodiments, the pharmaceutical composition reduces the adverse effects of a vaccine.

Exemplary Treatment

In some embodiments, the pharmaceutical compositions described herein is administered to a subject in need thereof. In some embodiments, the subject in need thereof has a condition or disease. In some embodiments, the pharmaceutical composition described herein is administered to treat a subject in need thereof with a condition or disease, wherein the pharmaceutical composition herein reduces a symptom or symptoms of the condition or disease. In some embodiments, the condition or disease is a viral infection. In some embodiments, the pharmaceutical composition is effective to at least partially reduce a viral load of a coronavirus.

In some embodiments, the viral infection is caused by a coronavirus. Exemplary examples of coronavirus can be, but not limited to, Alphacoronavirus, Betacoronavirus, a Gammacoronavirus, Deltacoronavirus, 229E coronavirus, NL63 coronavirus, OC43 coronavirus, HKU1 coronavirus, middle east respiratory syndrome related coronavirus (MERS-CoV), severe acute respiratory syndrome coronavirus (SARS-CoV), severe acute respiratory syndrome coronavirus 2 (SARS-CoV-2 (COVID-19)), a mutated form of any of the forgoing, a variant of any of the foregoing, or any combination thereof.

In some embodiments, subject has or was previously diagnosed with a general symptom of a coronavirus. In some embodiments, general symptoms of a viral infection can be, but not limited to, a fever, a cough, a shortness of breath, breathing difficulties, or any combination thereof.

Kits

Disclosed herein are kits. A kit can comprise a pharmaceutical composition described herein. In some aspects, a pharmaceutical composition can be packaged in a container. In some aspects, a kit can further comprise instructions that direct administration of a pharmaceutical composition to a subject. In some aspects, a kit can comprise a pharmaceutical composition disclosed herein and instructions for the use thereof.

Methods of making a kit can include placing a pharmaceutical composition described herein in a container for packaging. A method can further comprise an inclusion of instructions for use. In some cases, instructions for use can direct administration of a unit dose of a pharmaceutical composition to a subject

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A compound, or a pharmaceutically-acceptable salt or solvate thereof, having a structure represented by a structure of Formula (I):

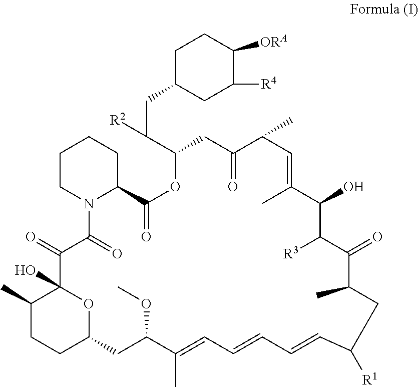

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R¹ is hydrogen or CH₃;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R⁴ is —OCH₃ or

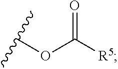

R⁵ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl;
   wherein when R¹ is hydrogen, at least one of (i) R² is not CH₃, (ii) R³ is not —OCH₃, or (iii) R⁴ is not —OCH₃;
and
   wherein when R¹ is CH₃, at least one of (i) R² is not CH₃, (ii) R³ is not hydrogen, —CH₃ or —OCH₃, or (iii) R⁴ is not —OCH₃.

Embodiment 2. The compound of Embodiment 1, wherein R⁴ is —OCH₃.

Embodiment 3. The compound of Embodiment 1 or 2, wherein R¹ is hydrogen or CH₃.

Embodiment 4. The compound of any one of Embodiments 1-3, wherein R¹ is hydrogen.

Embodiment 5. The compound of any one of Embodiments 1-4, wherein R² is CH₃.

Embodiment 6. The compound of any one of Embodiments 1-5, wherein: R¹ is hydrogen;

R² is CH₃;

R³ is hydrogen; and

R⁴ is —OCH₃.

Embodiment 7. The compound of any one of Embodiments 1-3, wherein R¹ is CH₃.

Embodiment 8. The compound of any one of Embodiments 1-3 or 7, wherein R² is hydrogen or CH₃.

Embodiment 9. The compound of any one of Embodiments 1-3 or 7-8, wherein R² is hydrogen.

Embodiment 10. The compound of any one of Embodiments 1-3 or 7-9, wherein: R¹ is CH₃;

R² is hydrogen;

R³ is —OCH₃; and

R⁴ is —OCH₃.

Embodiment 11. The compound of any one of Embodiments 1-3 or 7-9, wherein: R¹ is CH₃;

R² is hydrogen;

R³ is hydrogen; and

R⁴ is —OCH₃.

Embodiment 12. The compound of Embodiment 1, wherein R¹ is hydrogen, R² is CH₃, and R⁴ is —OCH₃.

Embodiment 13. The compound of Embodiment 1, wherein R¹ is an optionally substituted alkyl.

Embodiment 14. The compound of Embodiment 1, wherein R² is an optionally substituted alkyl.

Embodiment 15. The compound of Embodiment 1, wherein R³ is an optionally substituted alkoxy.

Embodiment 16. The compound of Embodiment 1, wherein R⁴ is an optionally substituted alkoxy.

Embodiment 17. A compound, or a pharmaceutically-acceptable salt or solvate thereof, having a structure represented by a structure of Formula (I-A):

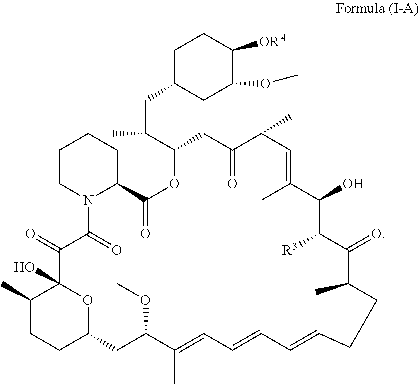

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰

R³ is hydrogen, halogen, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c}; or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the alkyl, cycloalkyl, heterocycloalkyl, aryl, and heteroaryl are each independently optionally substituted with one or more halogen, —OH, —NH₂, or C₁-C₆ alkyl; and
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl.

Embodiment 18. The compound of Embodiment 17, wherein R³ is hydrogen.

Embodiment 19. A compound, or a pharmaceutically-acceptable salt or solvate thereof, having a structure represented by a structure of Formula (I-B):

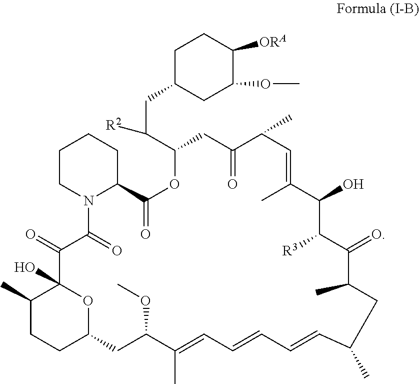

wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
   or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl;
and
wherein when R² is CH₃, R³ is not hydrogen, —CH₃ or —OCH₃.

Embodiment 20. The compound of Embodiment 19, wherein R² is hydrogen or CH₃.

Embodiment 21. The compound of Embodiment 19 or 20, wherein R² is hydrogen.

Embodiment 22. The compound of Embodiment 19 or 20, wherein R² is hydrogen and R³ is —OCH₃.

Embodiment 23. The compound of Embodiment 19 or 20, wherein R² is hydrogen and R³ is hydrogen.

Embodiment 24. The compound of Embodiment 19, wherein R² is an optionally substituted alkyl.

Embodiment 25. The compound of Embodiment 19, wherein R³ is an optionally substituted alkoxy.

Embodiment 26. A pharmaceutical composition comprising the compound of any one of the preceding Embodiments and at least one pharmaceutically-acceptable excipient.

Embodiment 27. The pharmaceutical composition of Embodiment 26, wherein the pharmaceutical composition is in a unit dosage form.

Embodiment 28. A method of treating a condition or disease in a subject in need thereof, comprising administering a pharmaceutical composition of Embodiments 26-27.

Embodiment 29. The method of Embodiment 28, wherein administering the pharmaceutical composition results in inhibiting mTORC1 and/or mTORC2.

Embodiment 30. The method of Embodiment 28 or 29, wherein administering the pharmaceutical composition further results in promoting immune cell differentiation.

Embodiment 31. The method of any one of Embodiments 28-30, wherein administering the pharmaceutical composition results in a suppression of proliferation of effector T-cells.

Embodiment 32. The method of any one of Embodiments 28-31, wherein administering the pharmaceutical composition further results in differentiation of memory T-cells.

Embodiment 33. The method of any one of Embodiments 28-32, wherein administering the pharmaceutical composition further results in differentiation of regulatory T-cells.

Embodiment 34. The method of any one of Embodiments 28-33, wherein administering the pharmaceutical composition comprises oral administration, rectally administration, parenterally administration, ocular administration, topical administration, intravenous administration, otic administration, inhalation administration, or any combination thereof.

Embodiment 35. The method of any one of Embodiments 28-34, wherein the condition or disease is a viral infection.

Embodiment 36. The method of Embodiment 35, wherein the viral infection is caused by a coronavirus.

Embodiment 37. The method of Embodiment 36, wherein the coronavirus is Alphacoronavirus, Betacoronavirus, a Gammacoronavirus, Deltacoronavirus, 229E coronavirus, NL63 coronavirus, OC43 coronavirus, HKU1 coronavirus, middle east respiratory syndrome related coronavirus (MERS-CoV), severe acute respiratory syndrome coronavirus (SARS-CoV), severe acute respiratory syndrome coronavirus 2 (SARS-CoV-2), a mutated form of any of these, or any combination thereof.

Embodiment 38. The method of Embodiment 28, wherein administering the pharmaceutical composition further comprises co-administration of a vaccine.

Embodiment 39. The method of Embodiment 38, wherein co-administration results in improved effectiveness of the vaccine.

Embodiment 40. The method of any one of Embodiments 35-39, wherein administering the pharmaceutical composition is effective to at least partially reduce a viral load of a coronavirus.

Embodiment 41. The method of any one of Embodiments 36-40, wherein the subject has or was previously diagnosed with a general symptom of a coronavirus.

Embodiment 42. The method of Embodiment 41, wherein the general symptom comprises a fever, a cough, a shortness of breath, breathing difficulties, or any combination thereof.

Embodiment 43. A kit comprising the pharmaceutical composition of Embodiment 26 or 27.

Embodiment 44. The kit of Embodiment 43, further comprising instructions for using the pharmaceutical composition.

Embodiment 45. The kit of Embodiment 43 or 44, further comprising a coronavirus vaccine.

Embodiment 46. The compound of Embodiment 1, wherein the compound of Formula (I), or pharmaceutically acceptable salt or solvate thereof, is selected from the group consisting of:

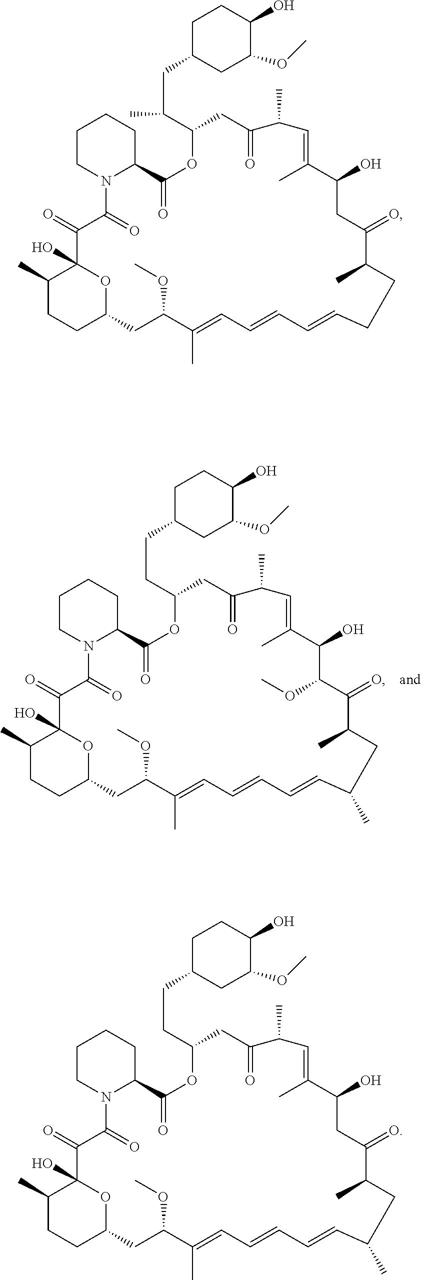

Embodiment 47. A method of treating a condition or disease in a subject in need thereof, comprising administering the compound of any one of Embodiments 1-25 or the pharmaceutical composition of any one of Embodiments 26-28, thereby treating the condition or disease in the subject.

Embodiment 48. Use of the compound of any one of Embodiments 1-25 or the pharmaceutical composition of any one of Embodiments 26-28 for the treatment of a condition or disease in a subject in need thereof, the use comprising administering to the subject the compound or the pharmaceutical composition, thereby treating the condition or disease in the subject.

Embodiment 49. Use of the compound of any one of Embodiments 1-25 or the pharmaceutical composition of any one of Embodiments 26-28 for the manufacture of a medicament for the treatment of a condition or disease in a subject in need thereof, the use comprising administering to the subject an effective amount of the compound or the pharmaceutical composition, thereby treating the condition or disease in the subject.

Embodiment 50. The method of any one of Embodiments 47-49, wherein administering the compound or the pharmaceutical composition results in inhibiting mTORC1 and/or mTORC2.

### EXAMPLES

The following examples are provided to further illustrate some embodiments of the present disclosure, but are not intended to limit the scope of the disclosure; it may be understood by their exemplary nature that other procedures, methodologies, or techniques known to those skilled in the art may alternatively be used.

### Example 1: Cell Studies of Rapamycin Analogs

### Compound Design and Production

In silico libraries were computationally generated of accessible rapamycin analogues and these were docked to FRB with and without FKBP12. The native rapamycin synthase in S. rapamycinicus was also engineered to produce these compounds. All engineered constructs were screened by PCR and be confirmed by sequencing. The compounds were synthesized in small-scale flask fermentations of the analyzed constructs and these products were rapidly confirmed using high-performance liquid chromatography (HPLC) coupled to tandem mass spectrometry (MS). Selected constructs were fermented at larger scale to produce sufficient titers for isolation, purification and structure determination via nuclear magnetic resonance (NMR) spectroscopy. Compounds were assessed for purity by HPLC and their structures were confirmed by high resolution MS (HRMS) and (NMR) experiments.

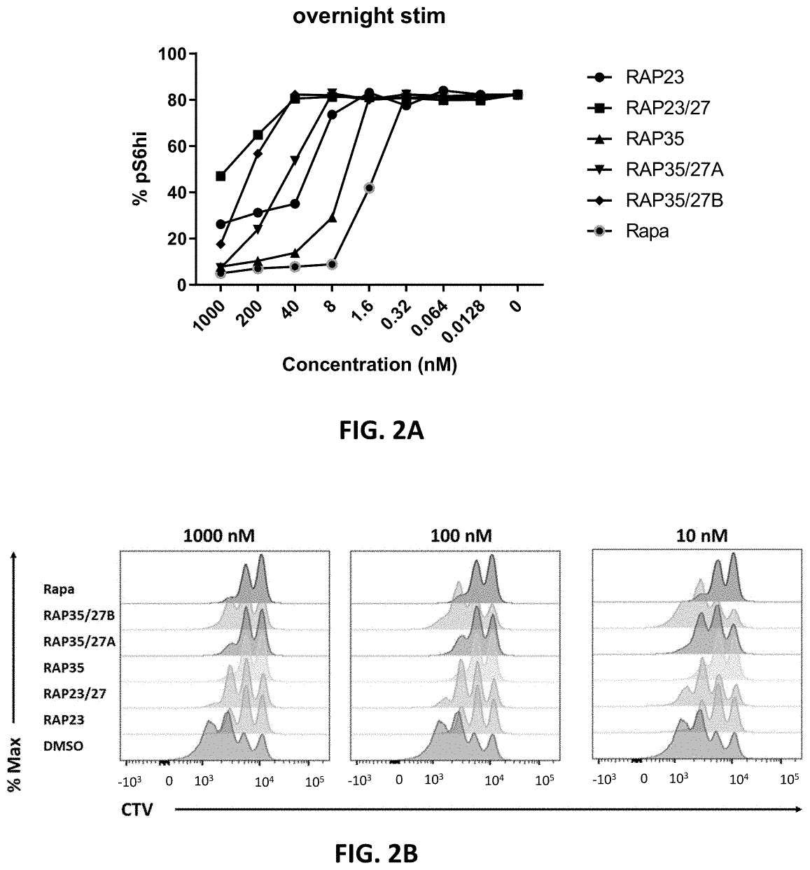

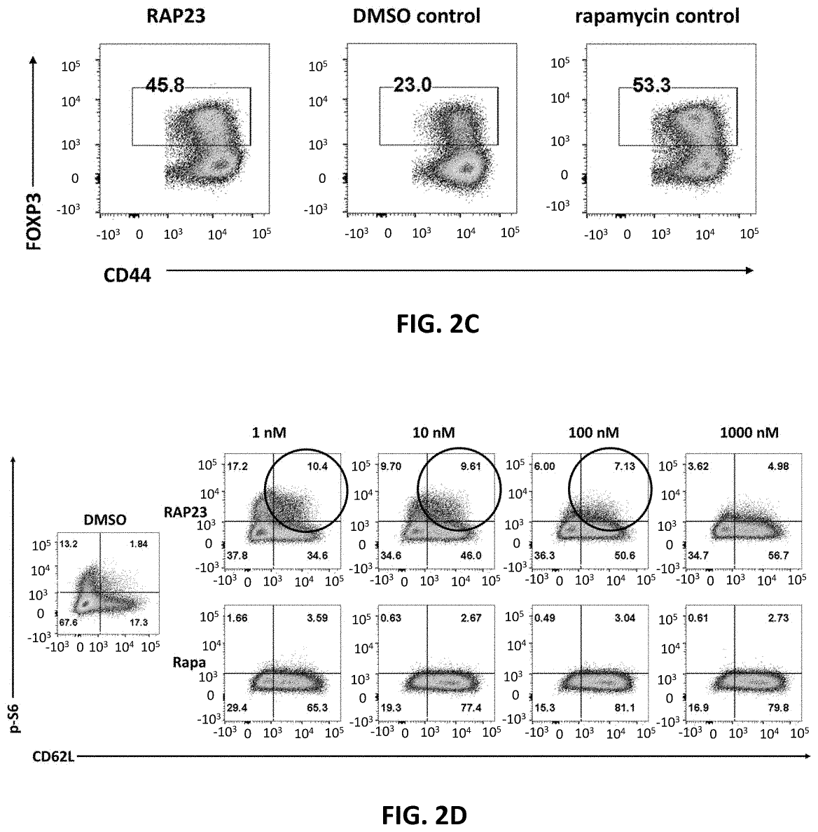

A set of five compounds (RAP23, RAP23/27, RAP35, RAP35/27A, and RAP35/27B), possessing alterations at individual and multiple locations, were designed to alter the specificity of competition with p70S6 kinase (S6K). These five compounds were synthesized and tested for the inhibition of TCR-induced mTOR activity, inhibition of proliferation, and their ability to promote FOXP3+ T-cells and CD8+ T-cell memory cells (FIG. 2).

The rapamycin analog RAP23 refers to chemical structure of (3S,6R,7E,9R,10R,12R,15E,17E,19E,21S,23S,26R,27R,34aS)-9,27-dihydroxy-3-((R)-1-((1S,3R,4R)-4-hydroxy-3-methoxycyclohexyl)propan-2-yl)-10,21-dimethoxy-6,8,12,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone or 23-desmethylrapamycin.

The rapamycin analog RAP23/27 refers to the chemical structure of (3S,6R,7E,9S,12R,15E,17E,19E,21S,23S,26R,27R,34aS)-9,27-dihydroxy-3-((R)-1-((1S,3R,4R)-4-hydroxy-3-methoxycyclohexyl)propan-2-yl)-21-methoxy-6,8,12,20,26-pentamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone or 23-desmethyl-27-demethoxyrapamycin.

The rapamycin analog RAP35 refers to the chemical structure of (3R,6R,7E,9R,10R,12R,14S,15E,17E,19E,21S,23S,26R,27R,34aS)-9,27-dihydroxy-3-(2-((1S,3R,4R)-4-hydroxy-3-methoxycyclohexyl)ethyl)-10,21-dimethoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone or 35-desmethylrapamycin.

The rapamycin analog RAP35/27A and RAP35/27B refers to the chemical structure of (3R,6R,7E,9S,12R,14S,15E,17E,19E,21S,23S,26R,27R,34aS)-9,27-dihydroxy-3-(2-((1S,3R,4R)-4-hydroxy-3-methoxycyclohexyl)ethyl)-21-methoxy-6,8,12,14,20,26-hexamethyl-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone or isomers of 23-desmethyl-27-demethoxyrapamycin.

### Results

Novel and reversible allosteric mTORC1 partial antagonists were computationally optimized using a structure-guided approach and synthesized for altered binding to the FRB domain of mTORC1 and FKBP12 via re-engineering of the pre-rapamycin polyketide synthase.

The five analogs (RAP23, RAP23/27, RAP35, RAP35/27A and RAP35/27B) and rapamycin were synthesized and their ability to inhibit mTOR activity upon activation of mouse T-cells (whole splenocyte from OT1 B6 mice) stimulated by soluble anti-CD3+ (3 μg/mL), anti-CD28 (2 μg/mL), and IgG (1.5 μg/mL) were tested using cell cytometry through employing phosphor-specific antibodies for p70S6K. FIG. 2A shows that following overnight stimulation, p70S6 high cells were significantly reduced at therapeutic concentrations (1-200 nM) of rapamycin and RAP35, but not for the other compounds.

The five analogs and rapamycin were assayed for their effect on proliferation and correlation to p70S6K inhibitory activity. CD8 cells isolated from TCR transgenic mice were labeled with CellTrace™ Violet (CTV) dye, activated anti-CD3+ (5 μg/mL) and anti-CD28 (2 μg/mL). The proliferation was measured by FACS. The five analogs were tested at varying concentrations (10 nM, 100 nM, and 100 μM), activated for 72 hours with rapamycin and DMSO treated controls. RAP23 never fully inhibited the p70S6 high signal, even at 1 uM. As predicted from its reduced S6K inhibition, FIG. 2B shows that RAP23 treated cells increased proliferation, relative to RAP35 and rapamycin.

The FACS analysis of naïve CD4+ mouse T cells stimulated with soluble anti-CD3+ (3 μg/mL) and anti-CD28 (2 μg/mL) in 1000 nM RAP23 and rapamycin for CD+4+CD25+FOXP3+ relative to uncreated DMSO control is shown in FIG. 2C. The RAP35 treated cells (1 uM) exhibited a strong effect on differentiation as the observed FOXP3+ count was 45.8 relative to rapamycin (53.3) at the same concentration.

RAP23 was further evaluated by treating naïve CD4+ T-cells at multiple concentrations (1 nM to 1 uM) for expansion of CD62L+ memory T-cells. The FACS analysis of naïve CD4+ mouse T-cells stimulated with soluble anti-CD3+ (3 μg/mL) and anti-CD28 (2 μg/mL) in the presence of increasing concentrations of RAP23 and rapamycin in CD8+CD6L2 relative to uncreated DMSO is shown in FIG. 2D. A significant portion of cells (7-10%) possessing a phospho-p70S6K high/CD62L+high phenotype was observed, in addition to a reduced phospho-p70S6K low/CD62L+high phenotype relative to all doses of rapamycin (including 1 nM). These 5 compounds were identified to have reduced immunosuppression (with improved safety) with maximum and durable expansion of memory T-cells. Thus, of these 5 initial compounds, RAP23 appeared to have the ability to promote potent generation of memory without potently inhibiting proliferation.

The compounds are further selected based on IC50 values for both 70S7K inhibition, inhibition of proliferation, and variable binding affinities to FKBP12. The FKBP12 binding measurements is employed as a predictor of pharmacokinetic half-life (T_{1/2}) in humans.

### Example 2: Engineering the Rapamycin Synthase and S. rapamycinicus for Production of Rapamycin Analogs

### Docking Results and Design Engineering

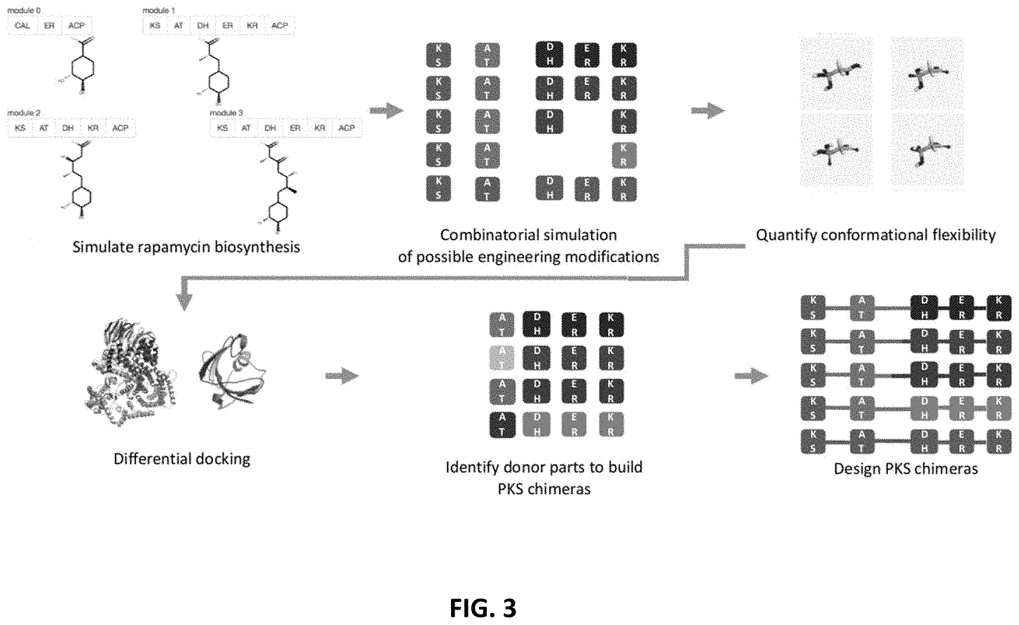

FIG. 3 displays the process for engineering and optimizing rapamycin analogs of the in silico library through computational techniques described herein. Six chemical libraries of potential engineered rapamycin analogues were generated, including nine simulated AT swaps at PKS modules 13 and 14, all possible single span entire module deletions between modules 2-9, removal of the rapM o-methylation, module 1 and 7 AT methylmalonyl-CoA to malonyl-CoA swaps to remove methyl groups, rapP swaps of all hydrophobic coding amino acids, and a rapJ (ketone) deletion. The highest mTOR/ternary ratio scoring compounds included modifying rapP to incorporate large amino acids (e.g. tyrosine, with a ratio of 0.62), and modifying module 14 via AT swap to include long side chains (e.g. an isobutyryl side group with a ratio of 0.59). With the predictions for the optimized rapamycin analogs in hand, the rapamycin synthase and rapamycin production organism (S. rapamycinicus) were then engineered to produce the desired optimized rapamycin analogs.

Medium and culturing optimization he culturing and fermentation conditions were optimized for the two rapamycin producing strains. The ISP-3 agar plate supplied the best sporulation condition among all the screened plates. On this plate, the wild-type strain (S. rapamycinicus ATCC29253) producing around 2*10¹⁰ spores per 100 mm Petri Dish after seven days.

### Genetic Manipulation of the Rapamycin-Producing Strain

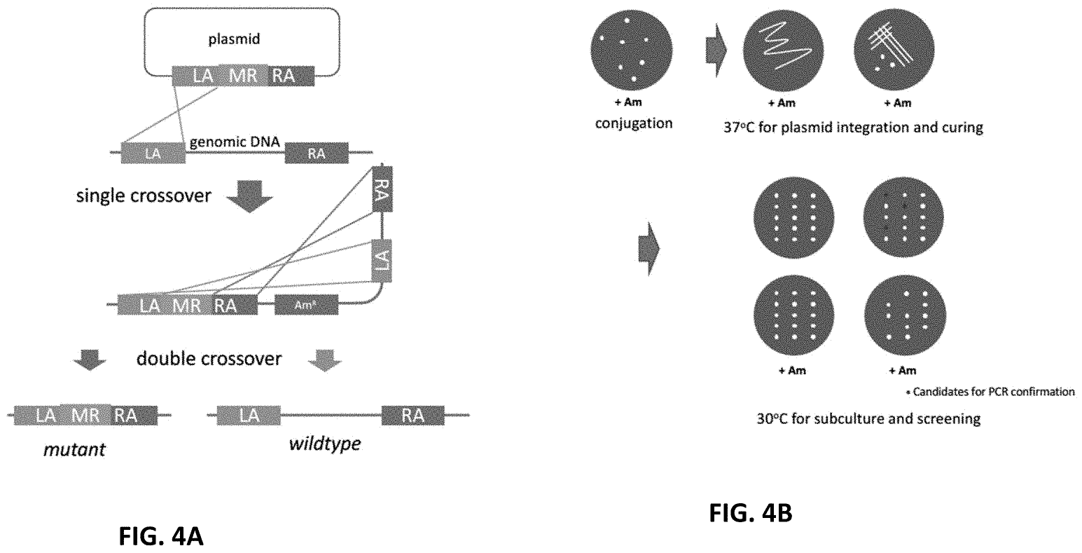

Rapamycin analogs were generated via the acyltransferase domain (AT) swap performed under the direction of rational design and docking. Modification of the rapamycin biosynthetic gene cluster (BGC) in the native producer was chosen over other means (e.g., refactoring the rapamycin synthase in a heterologous host), considering the large size and complexity of the rapamycin BGC. Genetic manipulation methods were established for the wild-type strain. For the DNA transfer method, a standard Streptomyces conjugation protocol was optimized, which resulted in high efficiency and reliable protocol for the wild-type strain. For the genomic DNA modification, a scar and marker-free method based on homologous recombination (FIG. 4A) was added to the method. In this method, a pKC1139-derived plasmid, which contains the target modified region (such as the donor AT domain) flanked by left and right arms, is first constructed. The plasmid was then conjugated into the acceptor strain, followed by integration, subculturing, and antibiotic screening (FIG. 4B). The screened colonies were subjected to PCR for genotype confirmation.

### Production of 45-Desmethylrapamycin Analogs

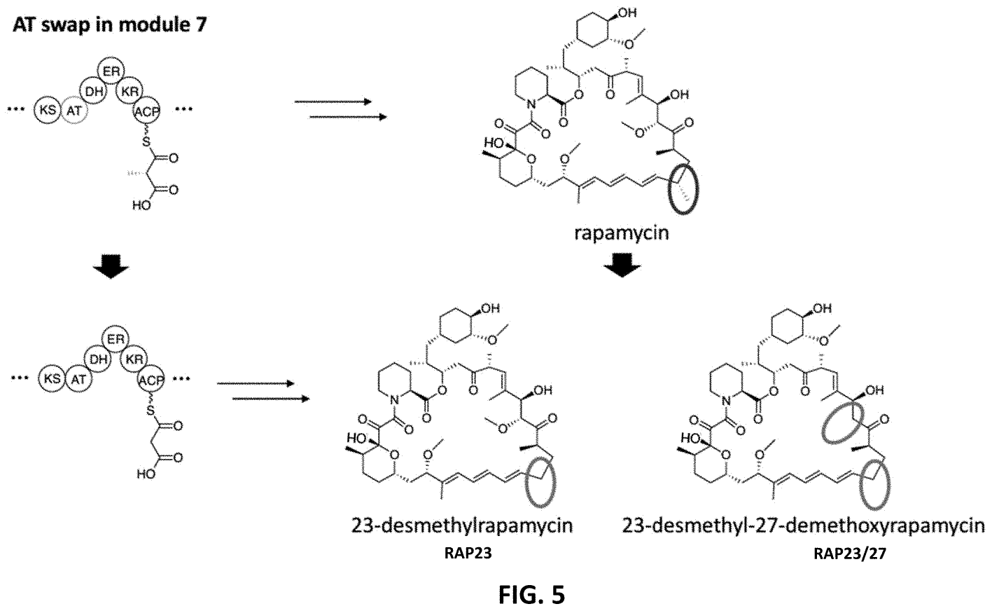

It was determined that the 23-methyl group maybe impact the interactions of mTORC1, rapamycin, and S6K1, and that removal of this methyl group may help to decrease the inhibitory effects that it has on S6K phosphorylation inhibition. Biosynthetically, this methyl group is introduced by the methylmalonyl-CoA specific acyltransferase (AT) domain in rapamycin PKS module 7. Exchanging the AT for one that specifies malonyl-CoA would remove the 23-methyl group in the final product. The DNA of Streptomyces was re-coded and synthesized to encode the AT domain in rapamycin PKS module 8. The corresponding plasmid harboring this AT-encoding DNA was constructed and conjugated into the wild-type strain, followed by integration, screening, and PCR confirmation. The positive strains were fermented using the rapamycin fermentation conditions determined above, and the resulting products were analyzed by HPLC-MS. Several new rapamycin analogs produced, and the two primary rapamycin analogs were isolated and purified to homogeneity. NMR, LC-MS, and UV-Vis analysis confirmed they are 23-desmethylrapamycin and 23-desmethyl-27-demethoxyrapmcin (FIG. 5).

### Production of 35-Desmethylrapamycin Analogs

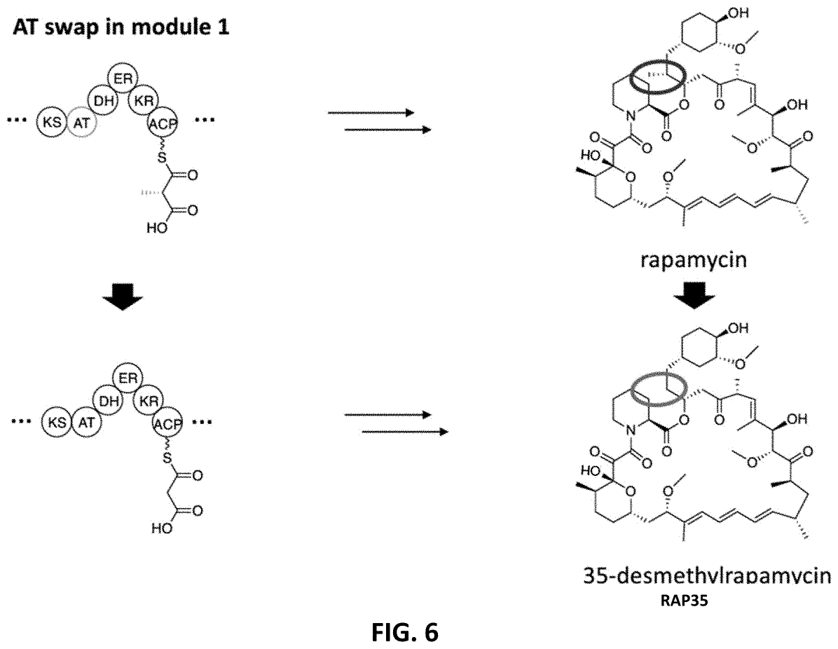

The 35-methyl group was predicted to be another possible effector of mTORC1, rapamycin, and S6K binding. The removal of this group was accomplished in the wild-type strain by exchanging the AT in rapamycin PKS module 1 to the re-coded AT of rapamycin PKS module 8. LC-MS analysis revealed several rapamycin analogs produced from the AT swapped strains. 35-desmethylrapamycin (FIG. 6) was synthesized and further confirmed based on molecular weight.

### Production of 9-Desmethylrapamycin Analogs

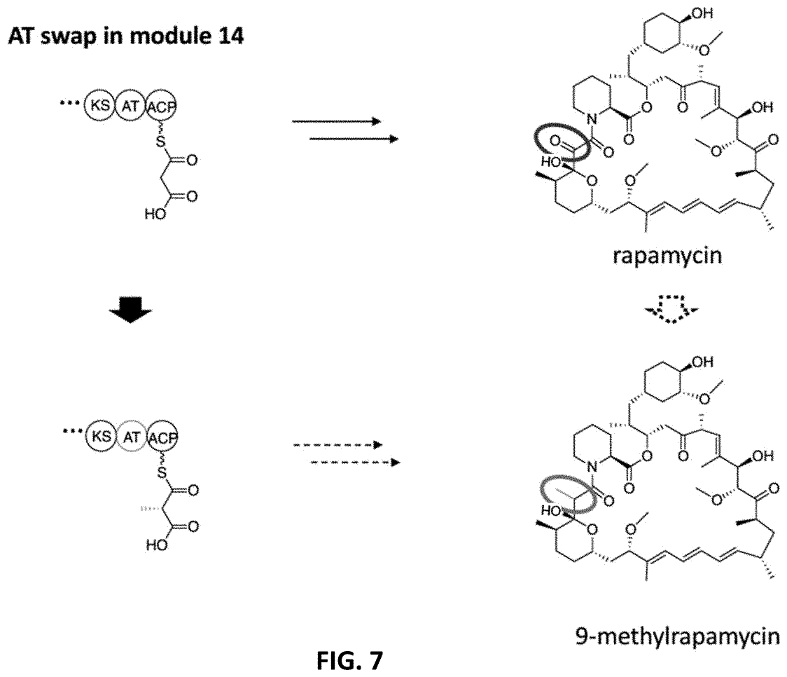

As the docking results revealed the highest mTOR/ternary ratio scoring compounds included 9-substituted rapamycin analogs, a strain to produce 9-methylrapamycin by replacing the AT in module 14, which is specific for malonyl-CoA, with one that is specific for methylmalonyl-CoA was constructed. AT swaps in the rapamycin PKS module 14 were performed in the wild-type strain. The AT of erythromycin PKS module 6, specific for malonyl-CoA, was selected as the donor AT based on the result of multiple sequence alignment (FIG. 7). The AT swaps were successfully conducted in the rapamycin synthase. The resulting strains were fermented using the rapamycin fermentation conditions developed as described above, but neither rapamycin nor its analogs were produced.

### Example 3: Production and Characterization of 23-Desmethylrapamycin (RAP23) and 23-Desmethyl-27-Demethoxyrapamycin (RAP23/27)

Generation of Plasmid pRP016

The following three fragments were first amplified using PCR:

at7_left_F
(SEQ ID NO: 1)
5′-GAATTCGAGCTCGGTACCCGGGGATCCTTCTAGAACGACGGCGTCT
TGGAGACCCT-3′
and
at7_left_R
(SEQ ID NO: 2)
5′-CGTACGTCGGAAGACTGACCCCGAAGGCCGACA-3′;

2-kb fragment from the genomic DNA of S. rapamycinicus ATCC 29253 using primers

at7_right_F
(SEQ ID NO: 3)
5′-CTTCGGGGTCAGTCTTCCGACGTACGCGTTCCAGC-3′
and
at7_right_R
(SEQ ID NO: 4)
5′-CAAGCTTGCATGCCTGCAGGTCGACTTCTAGAATTTCCCGGAAGCC
AGTGGTACGC-3′;

2.6-kb fragment from pUC19 using primers

at7-pUC19_F
(SEQ ID NO: 5)
5′-TCTAGAAGTCGACCTGCAGGCATG-3′
and
at7-pUC19_R
(SEQ ID NO: 6)
5′-TCTAGAAGGATCCCCGGGTACC-3′.

These three fragments were assembled to generate pRP008. Primers:

at7_backbone_F
(SEQ ID NO: 7)
5′-CTTCCGACGTACGCGTTCCAGC-3′
and
at7_backbone_R
(SEQ ID NO: 8)
5′-CCACTGACCCCGAAGGCCGACAC-3′

were used to linearize pRP008 by PCR.

The resulting 6.6 kb fragment was assembled with the synthesized gene block, rap_AT8, to generate pRP008. After validating the sequence of pRP008 by Sanger sequencing, the 5.2 kb fragment from XbaI-digested pRP008 was inserted into XbaI site of pKC1139 to afford pRP016.

Sequence of synthesized gene block rap_AT8:

(SEQ ID NO: 9)
GGTGTGTCGGCCTTCGGGGTCAGTGGTACGAACGCCCACGTCATCCTGGA
ATCGGCACCGCCGACCCAGCCCGCCGACAACGCGGTGATCGAGCGGGCGC
CGGAATGGCTGCCCATGGTGATCAGTGCACGTACGCAGTCCGCCCTGACC
GAACACGAAGGCCGGTTAAGAGCGTACCTGGCTGCCAGCCCGGGCGTGGA
TATGCGGGCCGTTGCATCGACGTTGGCCATGACACGGTCGGTGTTCGAGC
ACCGCGCTGTTCTGTTGGGTGACGACACCGTGACCGGCACCGCGGCGACC
GACCCGCGTGTCGTCTTCGTCTTCCCCGGTCAGGGTTCGCAGCGGGCTGG
GATGGGCGAAGAGTTGGCAGCAGCGTTCCCGGTGTTCGCTCGGATCCATC
AGCAGGTGTGGGACCTGCTGGATGTGCCGGATCTGGAGGTGAACGAGACC
GGGTACGCGCAGCCGGCGCTGTTCGCCTTGCAGGTCGCTCTGTTCGGGTT
GTTGGAGTCCTGGGGTGTTCGACCGGACGCGGTGGTCGGCCACAGTGTCG
GAGAACTGGCGGCCGGGTATGTCTCGGGTCTGTGGTCGCTGGAGGACGCC
TGCACGCTGGTGAGCGCAAGAGCACGGCTGATGCAGGCATTACCTGCTGG
TGGTGTGATGGTCGCGGTCCCGGTCTCCGAAGATGAGGCGAGGGCAGTTT
TAGGTGAGGGTGTCGAGATCGCCGCCGTGAACGGCCCCAGCAGCGTGGTG
CTGTCCGGCGACGAGGCTGCTGTCCTGCAGGCCGCTGAGGGGCTGGGGAA
GTGGACGAGATTAGCAACCAGCCACGCCTTCCACTCGGCCCGGATGGAGC
CGATGCTGGAGGAGTTCAGGACCGTCGCCGAGGGCCTGACGTACCGGACC
CCGCAGGTGTCGATGGCCGCGGGTGATCAGGTCACCACCACGGAGTACTG
GGTGCGGCAGGTTCGTGACACGGTCCGGTTCGGCGAGCAGGTGGCCTCGT
ACGAGGATGCGGTGTTCGTGGAGCTGGGTGCGGATCGCTCGTTGGCGAGA
CTGGTCGACGGCGTTGCGATGTTGCACGGCGATCACGAGGCCCAGGCGGC
TGTGTCGGCGTTGGCTCACCTGTACGTCAACGGTGTGACGGTGGACTGGC
CGGCATTGCTGGGAGATGCCCCGGCCACCCGGGTGCTGGACCTTCCGACG
TACGCGTTCCAGCACCAGC

Construction of the Engineered Strain S. rapamycinicus RAPA016

To introduce pRP016 into S. rapamycinicus ATCC 29253, conjugation between E. coli and Streptomyces was conducted. pRP016 was first transformed into E. coli ET12567, and the resulting single colony was inoculated into 2 mL LB medium containing kanamycin, chloramphenicol, and apramycin in a 5 mL round-bottom tube for growing overnight at 37° C. 0.5 mL of the overnight culture was inoculated into a 50 mL LB medium containing kanamycin, chloramphenicol, and apramycin in a 250 mL Erlenmeyer flask for growing at 37° C. until OD₆₀₀ reached 0.4. The culture was centrifuged at 4,000 g for 5 min. After removing the supernatant, the pellet cells were washed with 25 mL LB medium for three times. The washed cells were resuspended in 5 mL LB medium and put on ice. S. rapamycinicus ATCC 29253 spores stock containing 1×10⁹ number of spores in 20% glycerol aqueous solution was centrifuged at 2,300 g for 5 min. After removing the supernatant, the spores were washed with Difco 2×YT medium (BD, NJ) for three times. The spores were resuspended in 100 μL 2×YT medium, and the suspension was treated by heat shock at 50° C. for 10 min in a water broth. After cooling down to room temperature, 100 μL spore suspension was mixed with 100 μL washed E. coli cells suspension gently. The mixture was spread on dry Mannitol Soy (MS) agar plate evenly, and the cultures were incubated at 30° C. for 18 hours. 1 mL aqueous solution containing 1.5 mg nalidixic acid sodium salt and 0.75 mg apramycin, pH=7, was added on the grown cultures to overlay the whole plate evenly. After drying the plate surface in the biosafety cabinet, the culture was incubated at 30° C. until the conjugants appeared (5-7 days). The conjugants were streaked on SY plates (soluble starch 15 g, yeast extract 1 g, dipotassium phosphate 1 g, magnesium sulfate heptahydrate 1 g, sodium chloride 3 g, TES 6.87 g, difco agar 15 g, distilled water up to 1 L, pH=7.4) containing 25 mg/L nalidixic acid sodium salt and 25 mg/L apramycin. The streaked plate was first incubated at 30° C. for 24 h and then incubated at 37° C. until colonies appeared (5-7 days). These colonies were picked and streaked on new SY plates containing nalidixic acid sodium salt and apramycin, and the plates were incubated at 37° C. until colonies appeared (3 days). The resulting colonies were single crossover strains. To accelerate the double-crossover event and screen the resulting strains, the single crossover strains were grown on ISP Medium No. 3 (without antibiotics) at 30° C. for two generations. The resulting strains were streaked on ISP Medium No. 3, and the single colonies were screened by colony blotting on SY medium with/without apramycin. The colonies that lost apramycin resistance were subjected to a two-step diagnostic PCR for verification as follows: colony PCR was conducted using the primers:

at7_check1_F
(SEQ ID NO: 10)
5′-ACGACGGCGTCTTGGAGACCCT-3′
and
at7_check1_R
(SEQ ID NO: 11)
5′-ATTTCCCGGAAGCCAGTGGTACGC-3′

to afford 5.2 kb products containing the DNA sequence encoding the AT7 and its adjacent region on the genomic DNA of double-crossover strains.

The PCR products were then purified and used as the templates for the second-step PCR using the primer pair:

at8_check_F
(SEQ ID NO: 12)
5′-ACGTCATCCTGGAATC-3′
and
at8_check_R
(SEQ ID NO: 13)
5′-ACACCGTTGACGTACA-3′.

The colonies affording 1.1 kb PCR products are subjected to production and analysis.
Production and Analysis of S. rapamycinicus RAPA016

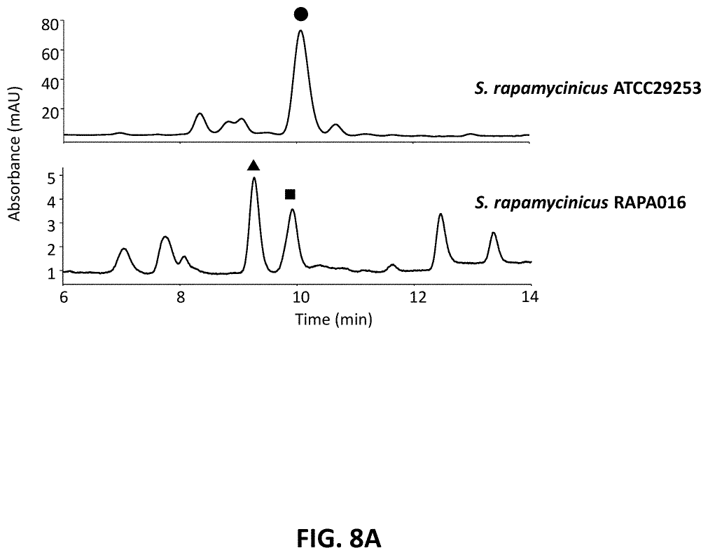

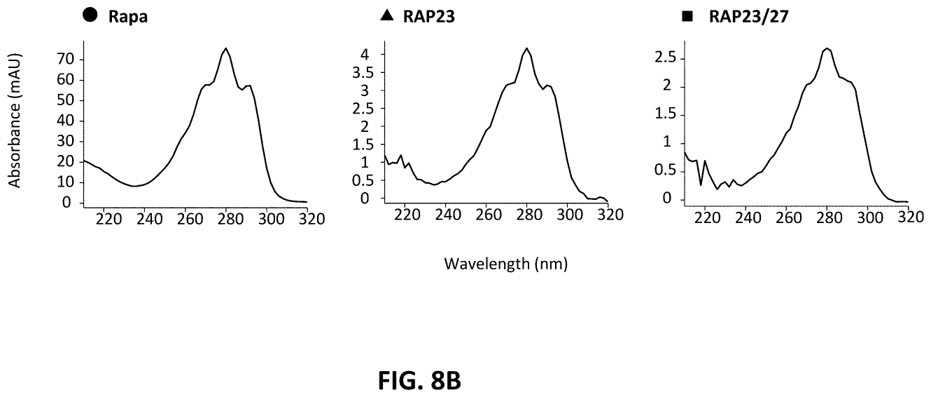

A single colony of S. rapamycinicus RAPA016 was spread on ISP Medium No. 3 to grow at 30° C. until sporulation (8-12 days). 1×1 cm of the culture on the medium was chopped into a 250 mL Erlenmeyer flask containing 25 mL of seed medium (malt extract 3 g, glucose 10 g, peptone 3 g, distilled water up to 1 L, pH=6.6) with 8-layer gauze cap. After growing at 28° C. for 4 days, 1.25 mL of the seed culture was inoculated into 25 mL of fermentation medium (soybean meal 15.4 g, potassium dihydrogen phosphate 5 g, distilled water up to 900 mL, pH=6.5. After autoclaving, 50 mL of 510 g/L mannose and 350 g/L L-Lysine hydrochloride were added, respectively) in a 250 mL Erlenmeyer flask with 8-layer gauze cap. The fermentation was conducted at 28° C., 220 rpm for 4 days. 200 μL of methanol was added into 100 μL of well mixed fermentation broth. After vigorously shaking by vortex for 30 min at room temperature, the mixture was centrifuged at 15000 g for 5 min. 20 μL of the supernatant was subjected to HPLC-DAD analysis on an Agilent Zorbax column (SB-C18, 5 m, 4.6×250 mm, Agilent Technologies Inc., CA) heated to 50° C. by gradient elution of solvent A (H₂O containing 0.1% formic acid) and solvent B (CH₃CN containing 0.1% formic acid) at a flow rate of 1 mL/min as following program: T=0, 30% B; T=2 min, 30% B; T=15 min, 95% B; T=20 min, 95% B; T=21 min, 30% B; T=26 min, 30% B. The resulting HPLC chromatography showed that S. rapamycinicus RAPA016 cannot produce rapamycin. Instead, two new peaks having similar UV absorption spectra to rapamycin (FIG. 8), which has triple UV absorption peaks at 270-290 nm wavelength derived from the triene structure, were detected. LC-MS/MS analysis indicated these two new peaks were supposed to be derived from 23-desmethylrapamycin (RAP23) and 23-desmethyl-27-demethoxyrapamycin (RAP23/27), as they have 14 Da and 44 Da less than rapamycin. Moreover, the fragmentation of their sodium adducts also gave the expected ions.

To accumulate 23-desmethylrapamycin (RAP23) and 23-desmethyl-27-demethoxyrapamycin (RAP23/27) for characterization and bioactivity assays, a 7.2-L scale of fermentation was conducted. 7.2 L fermentation medium was prepared and aliquoted into eighteen 2 L Erlenmeyer flasks. 20 mL of the seed culture was inoculated into each 2 L Erlenmeyer flask, which was then capped with 8-layer gauze. The fermentation was conducted at 28° C., 220 rpm for 4 days. To process the culture, the fermentation broth was centrifuged at 9,000 g for 10 min at room temperature, and the cell pellet was collected and combined. After transferring the cells into a 5 L glass beaker, 2 volumes of acetonitrile were added into 1 volume of the cells. The mixture was stirred vigorously using a magnetic stirrer at room temperature for 1 h. The mixing was halted, and the mixture was allowed to settle under gravity for 30 min. The solvent layer was poured into a 5 L Erlenmeyer flask, and another 2 volumes of acetonitrile was added into the cells for extraction. After three times of extraction in total, the solvent layers were combined and concentrated under reduced pressure. To purify 23-desmethylrapamycin (RAP23) and 23-desmethyl-27-demethoxyrapamycin (RAP23/27), the residue was dissolved in ethyl acetate, and silica gel was added into the solution. After removing ethyl acetate under reduced pressure, the dried powder was loaded onto the top of a silica gel 60 (230-400 mesh, Alfa Aesar, MA) open column (30 mm O.D.×300 mm L). The column was eluted with chloroform followed by an increasing methanol concentration to 10% in 1% steps, in which 500 mL of solvent was used for each step. The fractions were analyzed using HPLC-DAD, and those containing the target compounds were combined and concentrated under reduced pressure. The residue was dissolved in methanol and loaded onto the top of a Sephadex LH20 (GE Healthcare, IL) open column (19 mm O.D.×813 mm L). The column was eluted with methanol at a flow rate of 15 mL/hour, and the fractions were collected every 2 mL. The fractions containing 23-desmethylrapamycin (RAP23) and 23-desmethyl-27-demethoxyrapamycin (RAP23/27) were combined and concentrated under reduced pressure. The crude was then dissolved in acetonitrile, and the resulting solution was subjected to semi-preparation using an Agilent Zorbax column (SB-C18, 5 μm, 9.4×250 mm, Agilent Technologies Inc., CA) heated to 50° C. by isocratic elution of 65% acetonitrile with 35% water at a flow rate of 3.5 mL/min. The absorbance at 278 nm wavelength was monitored. The following fractions were collected and dried under reduced pressure, respectively: 20.5 min to 23 min for 23-desmethylrapamycin (RAP23) (6.6 mg), 24 min to 26 min for 23-desmethyl-27-demethoxyrapamycin (RAP23/27) (7.4 mg). The purified 23-desmethylrapamycin (RAP23) and 23-desmethyl-27-demethoxyrapamycin (RAP23/27) were dissolved in acetonitrile-d₃ and subjected to NMR experiments via ¹H, ¹³C, COSY, DEPT-135, HSQC, and HMBC. The resulting NMR spectra enabled the assignment of the proton and carbon shift of the major rotamer of 23-desmethylrapamycin (RAP23) and 23-desmethyl-27-demethoxyrapamycin (RAP23/27). For 23-desmethylrapamycin (RAP23), two geminally coupled hydrogen atoms were observed attached on 23-C instead of a methyl group and a hydrogen atom on this position in rapamycin. These two hydrogen atoms were coupled with hydrogen atoms on neighbor carbons, observed in COSY spectrum, and adjacent carbon atoms, observed in HMBC spectrum. For 23-desmethyl-27-demethoxyrapamycin (RAP23/27), other than the hydrogen atom substituting of methyl group on 23-C, two geminally coupled hydrogen atoms were observed attached on 27-C instead of a methoxy group and a hydrogen atom on this position in rapamycin. The correlations with neighbor hydrogen atoms and adjacent carbon atoms were also observed in COSY and HMBC spectra. The assignments of proton and carbon shifts, as well as important NMR correlations are summarized in Table 1 and Table 2.

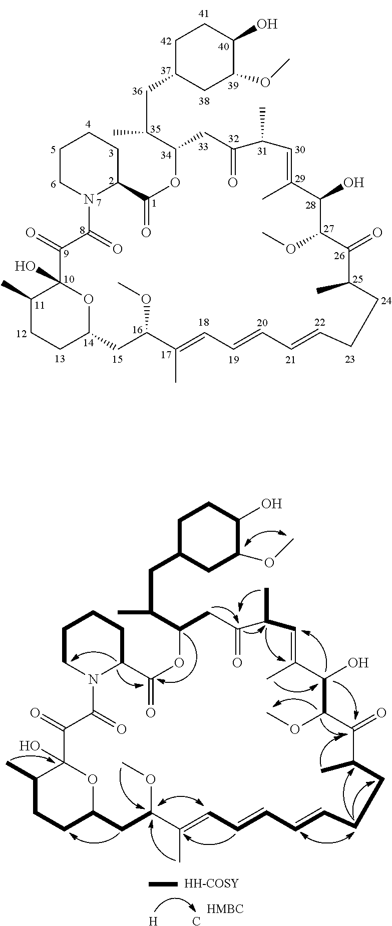

TABLE 1
¹H and ¹³C NMR signals assignment and important NMR correlations
of 23-desmethylrapmycin (RAP23) (rotamer of trans form at the amide
bond, in acetonitrile-d3)

| Position | ¹H shift (ppm) | ¹³C shift (ppm) |
|---|---|---|
| 1 | | 170.49 |
| 2 | 5.14 | 52.43 |
| 3 | 1.68, 2.29 | 27.54 |
| 4 | 1.45, 1.73 | 21.64 |
| 5 | 1.38, 1.64 | 25.83 |
| 6 | 3.22, 3.47 | 45.29 |
| 8 | | 167.89 |
| 9 | | 198.03 |
| 10 | | 99.76 |
| 11 | 2.03 | 35.3 |
| 11-CH₃ | 0.85 | 16.37 |
| 12 | 1.56, 1.62 | 27.71 |
| 13 | 1.28, 1.78 | 31.39 |
| 14 | 3.93 | 68.26 |
| 15 | 1.33, 1.85 | 32.95 |
| 16 | 3.68 | 84.13 |
| 16-OCH₃ | 3.1 | 56.22 |
| 17 | | 138.6 |
| 17-CH₃ | 1.65 | 10.81 |
| 18 | 6.09 | 128.87 |
| 19 | 6.42 | 127.95 |
| 20 | 6.32 | 133.8 |
| 21 | 6.18 | 133.44 |
| 22 | 5.66 | 134.47 |
| 23 | 2.12, 2.29 | 30.04 |
| 24 | 1.23, 1.67 | 32.47 |
| 25 | 2.53 | 41.38 |
| 25-CH₃ | 0.87 | 13.18 |
| 26 | | 212.39 |
| 27 | 4.04 | 86.12 |
| 27-OCH₃ | 3.26 | 58.18 |
| 28 | 4.17 | 77.43 |
| 29 | | 137.52 |
| 29-CH₃ | 1.8 | 14.65 |
| 30 | 5.26 | 125.89 |
| 31 | 3.32 | 46.89 |
| 31-CH₃ | 0.95 | 16.25 |
| 32 | | 209.48 |
| 33 | 2.52, 2.67 | 41.43 |
| 34 | 5.13 | 75.58 |
| 35 | 1.8 | 34.84 |
| 35-CH₃ | 0.85 | 15.61 |
| 36 | 1.06, 1.14 | 39.66 |
| 37 | 1.38 | 33.82 |
| 38 | 0.63, 2.03 | 35.83 |
| 39 | 2.86 | 85.23 |
| 39-OCH₃ | 3.35 | 57.15 |
| 40 | 3.24 | 74.73 |
| 41 | 1.23, 1.85 | 32.98 |
| 42 | 0.94, 1.62 | 32.13 |

TABLE 2
¹H and ¹³C NMR signals assignment and important NMR correlations of 23-desmethyl-
27-demethoxy-rapmycin (RAP23/27) (rotamer of trans form at the amide bond, in acetonitrile-d3)

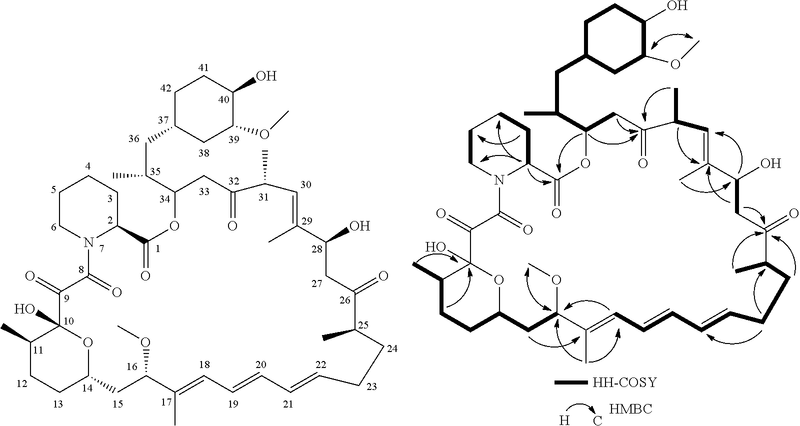

| Position | ¹H shift (ppm) | ¹³C shift (ppm) |
|---|---|---|
| 1 | | 170.3 |
| 2 | 5.10 | 52.58 |
| 3 | 1.65, 2.25 | 27.50 |
| 4 | 1.39, 1.73 | 21.68 |
| 5 | 1.39, 1.63 | 25.74 |
| 6 | 3.14, 3.47 | 45.21 |
| 8 | | 167.88 |
| 9 | | 198.54 |
| 10 | | 99.83 |
| 11 | 2.02 | 35.3 |
| 11-CH₃ | 0.86 | 16.39 |
| 12 | 1.55, 1.62 | 27.8 |
| 13 | 1.27, 1.77 | 31.64 |
| 14 | 3.93 | 68.38 |
| 15 | 1.41, 1.84 | 33.26 |
| 16 | 3.64 | 84.28 |
| 16-OCH₃ | 3.09 | 56.13 |
| 17 | | 138.05 |
| 17-CH₃ | 1.64 | 10.87 |
| 18 | 6.04 | 129.29 |
| 19 | 6.39 | 127.68 |
| 20 | 6.22 | 134.08 |
| 21 | 6.14 | 132.58 |
| 22 | 5.55 | 135.71 |
| 23 | 2.05, 2.25 | 31.5 |
| 24 | 1.43, 1.84 | 32.3 |
| 25 | 2.51 | 46.03 |
| 25-CH₃ | 0.98 | 16.73 |
| 26 | | 214.72 |
| 27 | 2.53, 2.64 | 48.59 |
| 28 | 4.3 | 72.56 |
| 29 | | 141.14 |
| 29-CH₃ | 1.62 | 13.56 |
| 30 | 5.29 | 124.72 |
| 31 | 3.32 | 46.75 |
| 31-CH₃ | 1.03 | 16.37 |
| 32 | | 209.28 |
| 33 | 2.62, 2.71 | 41.74 |
| 34 | 5.22 | 75.2 |
| 35 | 1.84 | 34.49 |
| 35-CH₃ | 0.86 | 15.82 |
| 36 | 1.06, 1.15 | 39.61 |
| 37 | 1.39 | 33.85 |
| 38 | 0.63, 2.05 | 35.58 |
| 39 | 2.87 | 85.31 |
| 39-OCH₃ | 3.34 | 57.11 |
| 40 | 3.26 | 74.71 |
| 41 | 1.24, 1.84 | 32.97 |
| 42 | 0.93, 1.62 | 32.17 |

### Example 4: Production and Characterization of 35-Desmethylrapamycin (RAP35) and 35-Desmethyl-27-Demethoxyrapamycin (RAP35/27)

Generation of the Plasmid pRP060

The following fragments were first amplified using PCR:

2-kb fragment from the genomic DNA of S. rapamycinicus ATCC 29253 using primers:

at1_left_F
(SEQ ID NO: 14)
5′-ACCATGATTACGCCTCTAGAGGGTCGACACGGCGGTCTGT-3′
and
at1_left_R
(SEQ ID NO: 15)
5′-ATGACGTGGGCGTTCGTACCGCTGACCCCGAAGGCCGACA-3′;

2-kb fragment from the genomic DNA of S. rapamycinicus ATCC 29253 using primers:

at1_right_F
(SEQ ID NO: 16)
5′-CGGCCACCCGGGTGCTGGACCTTCCGACGTACGCGTTCCA-3′
and
at1_right_R
(SEQ ID NO: 17)
5′-AAACGACGGCCAGTTCTAGAAAGGTTTCCTGGAAACTCAGTGTG-
3′;

2.6-kb fragment from pUC19 using primers:

at1-pUC19_F
(SEQ ID NO: 18)
5′-TTCCAGGAAACCTTTCTAGAACTGGCCGTCGTTTTACAAC-3′
and
at1-pUC19_R
(SEQ ID NO: 19)
5′-CGCCGTGTCGACCCTCTAGAGGCGTAATCATGGTCATAGC-3′;

1.2 kb fragment from pRP008 using the primers:

at1-at8_F
(SEQ ID NO: 20)
5′-TGTCGGCCTTCGGGGTCAGCGGTACGAACGCCCACGTCATCC
TG-3′
and
at1-at8_R
(SEQ ID NO: 21)
5′-TGGAACGCGTACGTCGGAAGGTCCAGCACCCGGGTGGCCG-3′.

These four fragments were assembled to generate pRP058. After validating the sequence of pRP058 by Sanger sequencing, the 5.2 kb fragment from XbaI-digested pRP058 was inserted into XbaI site of pKC1139 to afford pRP060.

Construction of the Engineered Strain S. rapamycinicus RAPA060

To introduce pRP060 into S. rapamycinicus ATCC 29253, conjugation between E. coli and Streptomyces was conducted. pRP060 was first transformed into E. coli ET12567, and the resulting single colony was inoculated into 2 mL LB medium containing kanamycin, chloramphenicol, and apramycin in a 5 mL round-bottom tube for growing overnight at 37° C. 0.5 mL of the overnight culture was inoculated into a 50 mL LB medium containing kanamycin, chloramphenicol, and apramycin in a 250 mL Erlenmeyer flask for growing at 37° C. until OD₆₀₀ reached 0.4. The culture was centrifuged at 4,000 g for 5 min. After removing the supernatant, the pellet cells were washed with 25 mL LB medium for three times. The washed cells were resuspended in 5 mL LB medium and put on ice. S. rapamycinicus ATCC 29253 spores stock containing 1×10⁹ number of spores in 20% glycerol aqueous solution was centrifuged at 2,300 g for 5 min. After removing the supernatant, the spores were washed with Difco 2×YT medium (BD, NJ) for three times. The spores were resuspended in 100 μL 2×YT medium, and the suspension was treated by heat shock at 50° C. for 10 min in a water broth. After cooling down to room temperature, 100 μL spore suspension was mixed with 100 μL washed E. coli cells suspension gently. The mixture was spread on dry Mannitol Soy (MS) agar plate evenly, and the cultures were incubated at 30° C. for 18 hours. 1 mL aqueous solution containing 1.5 mg nalidixic acid sodium salt and 0.75 mg apramycin, pH=7, was added on the grown cultures to overlay the whole plate evenly. After drying the plate surface in the biosafety cabinet, the culture was incubated at 30° C. until the conjugants appeared (5-7 days). The conjugants were streaked on SY plates (soluble starch 15 g, yeast extract 1 g, dipotassium phosphate 1 g, magnesium sulfate heptahydrate 1 g, sodium chloride 3 g, TES 6.87 g, difco agar 15 g, distilled water up to 1 L, pH=7.4) containing 25 mg/L nalidixic acid sodium salt and 25 mg/L apramycin. The streaked plate was first incubated at 30° C. for 24 h and then incubated at 37° C. until colonies appeared (5-7 days). These colonies were picked and streaked on new SY plates containing nalidixic acid sodium salt and apramycin, and the plates were incubated at 37° C. until colonies appeared (3 days). The resulting colonies were single crossover strains. To accelerate the double-crossover event and screen the resulting strains, the single crossover strains were grown on ISP Medium No. 3 (without antibiotics) at 30° C. for two generations. The resulting strains were streaked on ISP Medium No. 3, and the single colonies were screened by colony blotting on SY medium with/without apramycin. The colonies that lost apramycin resistance were subjected to a two-step diagnostic PCR for verification as follows: colony PCR was conducted using the primers:

at1_left_F and

at1_right_R

to afford 5.2 kb products containing the DNA sequence encoding the AT7 and its adjacent region on the genomic DNA of double-crossover strains. The PCR products were then purified and used as the templates for the second-step PCR using the primer pair:
a. at8_check_F and

b. at8_check_R.

The colonies affording 1.1 kb PCR products are subjected to production analysis.
Production and Analysis of S. rapamycinicus RAPA060

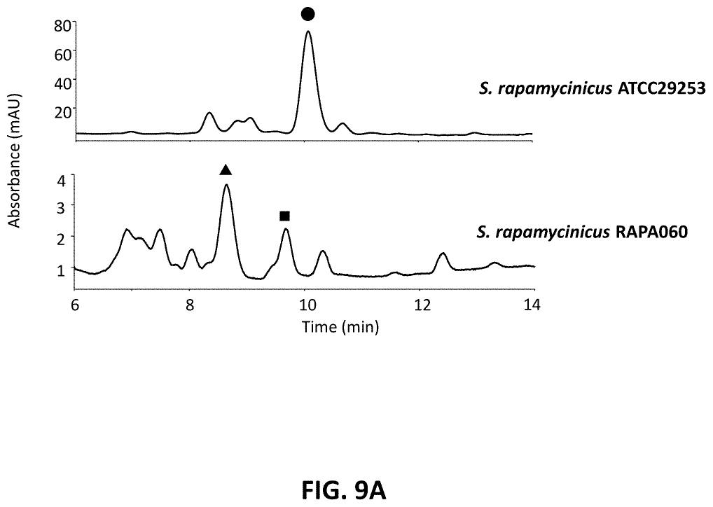

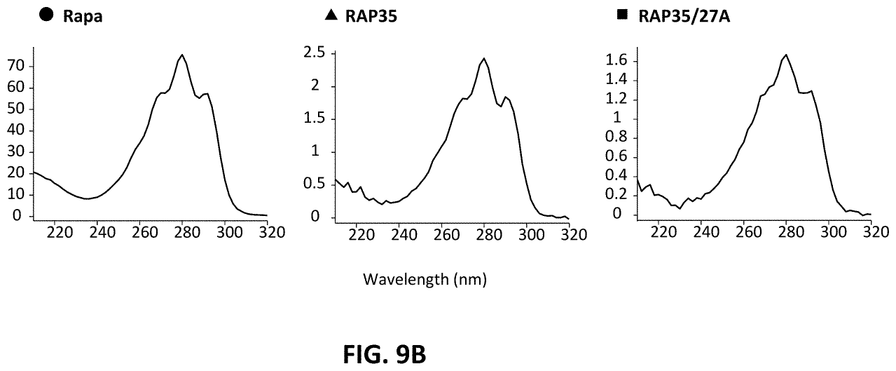

A single colony of S. rapamycinicus RAPA060 was spread on ISP Medium No. 3 to grow at 30° C. until sporulation (8-12 days). 1×1 cm of the culture on the medium was chopped into a 250 mL Erlenmeyer flask containing 25 mL of seed medium (malt extract 3 g, glucose 10 g, peptone 3 g, distilled water up to 1 L, pH=6.6) with 8-layer gauze cap. After growing at 28° C. for 4 days, 1.25 mL of the seed culture was inoculated into 25 mL of fermentation medium (soybean meal 15.4 g, potassium dihydrogen phosphate 5 g, distilled water up to 900 mL, pH=6.5. After autoclaving, 50 mL of 510 g/L mannose and 350 g/L L-Lysine hydrochloride were added, respectively) in a 250 mL Erlenmeyer flask with 8-layer gauze cap. The fermentation was conducted at 28° C., 220 rpm for 4 days. 200 μL of methanol was added into 100 μL of well mixed fermentation broth. After vigorously sharking by vortex for 30 min at room temperature, the mixture was centrifuged at 15000 g for 5 min. 20 μL of the supernatant was subjected to HPLC-DAD analysis on an Agilent Zorbax column (SB-C18, 5 m, 4.6×250 mm, Agilent Technologies Inc., CA) heated to 50° C. by gradient elution of solvent A (H₂O containing 0.1% formic acid) and solvent B (CH₃CN containing 0.1% formic acid) at a flow rate of 1 mL/min as following program: T=0, 30% B; T=2 min, 30% B; T=15 min, 95% B; T=20 min, 95% B; T=21 min, 30% B; T=26 min, 30% B. The resulting HPLC trace showed that S. rapamycinicus RAPA060 cannot produce rapamycin. Instead, two new peaks having similar UV-VIS absorption spectra to rapamycin (FIG. 9) were detected. LC-MS/MS analysis indicated these two new peaks were supposed to be derived from 35-desmethylrapamycin (RAP35) and 35-desmethyl-27-demethoxyrapamycin (RAP35/27), as they have 14 Da and 44 Da less than rapamycin. Moreover, the fragmentation of their sodium adducts also gave the expected ions. Large-scale fermentation and isolation of 35-desmethylrapamycin (RAP35) and 35-desmethyl-27-demethoxyrapamycin (RAP35/27).

To accumulate 35-desmethylrapamycin (RAP35) and 35-desmethyl-27-demethoxyrapamycin (RAP35/27) for characterization and bioactivity assays, a 7.2-L scale of fermentation was conducted. 7.2 L fermentation medium was prepared and aliquoted into eighteen 2 L Erlenmeyer flasks. 20 mL of the seed culture was inoculated into each 2 L Erlenmeyer flask, which was then capped with 8-layer gauze. The fermentation was conducted at 28° C., 220 rpm for 4 days. To process the culture, the fermentation broth was centrifuged at 9,000 g for 10 min at room temperature, and the cell pellet was collected and combined. After transferring the cells into a 5 L glass beaker, 2 volumes of acetonitrile were added into 1 volume of the cells. The mixture was stirred vigorously using a magnetic stirrer at room temperature for 1 h. The mixing was halted, and the mixture was allowed to settle under gravity for 30 min. The solvent layer was poured into a 5 L Erlenmeyer flask, and another 2 volumes of acetonitrile was added into the cells for extraction. After three times of extraction in total, the solvent layers were combined and concentrated under reduced pressure. To purify 35-desmethylrapamycin (RAP35) and 35-desmethyl-27-demethoxyrapamycin (RAP35/27), the residue was dissolved in methanol and loaded onto the top of a Sephadex LH20 (GE Healthcare, IL) open column (19 mm O.D.×813 mm L). The column was eluted with methanol at a flow rate of 15 mL/hour, and the fractions were collected every 2 mL. After analysis using HPLC-DAD, the fractions containing 35-desmethylrapamycin (RAP35) and 35-desmethyl-27-demethoxyrapamycin (RAP35/27) were combined and concentrated under reduced pressure. The crude was then dissolved in acetonitrile, and the resulting solution was subjected to semi-preparation using an Agilent Zorbax column (SB-C18, 5 μm, 9.4×250 mm, Agilent Technologies Inc., CA) heated to 50° C. by isocratic elution of 65% acetonitrile with 35% water at a flow rate of 3.5 mL/min. The absorbance at 278 nm wavelength was monitored. The following fractions were collected and dried under reduced pressure, respectively: 16.5 min to 18 min for 35-desmethylrapamycin (RAP35) (10.2 mg), 21.5 min to 22 min for 35-desmethyl-27-demethoxyrapamycin (RAP35/27) (5.4 mg). In the process of semi-preparation, 35-desmethyl-27-demethoxyrapamycin (RAP35/27) tended to be unstable, as approximately one fifth of the compound transformed to a more nonpolar compound spontaneously, revealed by HPLC analysis. LC-MS/MS analysis indicated that the newly formed compound has same molecular weight and similar MS/MS fragmentation to 35-desmethyl-27-demethoxyrapamycin (RAP35/27A). The newly formed compound is considered to be an isomer of 35-desmethyl-27-demethoxyrapamycin (RAP35/27B).

The purified 35-desmethylrapamycin (RAP35) and 35-desmethyl-27-demethoxyrapamycin (RAP35/27A) were dissolved in acetonitrile-d₃ and subjected to NMR experiments via ¹H, ¹³C, COSY, DEPT-135, HSQC, and HMBC. The resulting NMR spectrums enabled the assignment of the proton and carbon shift of the major rotamer of 35-desmethylrapamycin (RAP35) and 35-desmethyl-27-demethoxyrapamycin (RAP35/27A) (the major isomer). For 35-desmethylrapamycin (RAP35), two geminally coupled hydrogen atoms were observed attached on 35-C instead of a methyl group and a hydrogen atom on this position in rapamycin. These two hydrogen atoms were coupled with hydrogen atoms on neighbor carbons, observed in COSY spectrum, and adjacent carbon atoms, observed in HMBC spectrum. For 35-desmethyl-27-demethoxyrapamycin (RAP35/27), other than the hydrogen atom substituting of methyl group on 35-C, two geminally coupled hydrogen atoms were observed attached on 27-C instead of a methoxy group and a hydrogen atom on this position in rapamycin. The correlations with neighbor hydrogen atoms and adjacent carbon atoms were also observed in COSY and HMBC spectra. The assignments of proton and carbon shifts, as well as important NMR correlations are summarized in Table 3 and Table 4.

TABLE 3
¹H and ¹³C NMR signals assignment of 35-desmethylrapmycin (RAP35) (rotamer of
trans form at the amide bond, in acetonitrile-d3)

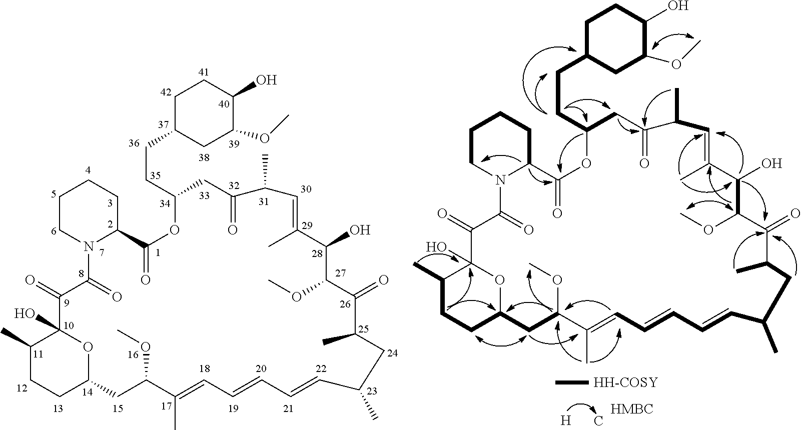

| Position | ¹H shift (ppm) | ¹³C shift (ppm) |
|---|---|---|
| 1 | | 170.3 |
| 2 | 5.06 | 52.96 |
| 3 | 1.68, 2.23 | 27.76 |
| 4 | 1.39, 1.7 | 21.99 |
| 5 | 1.37, 1.62 | 26.02 |
| 6 | 3.17, 3.46 | 45.81 |
| 8 | | 168.33 |
| 9 | | 198.3 |
| 10 | | 100.27 |
| 11 | 2.05 | 36.03 |
| 11-CH₃ | 0.84 | 16.66 |
| 12 | 1.58, 1.63 | 27.95 |
| 13 | 1.29, 1.86 | 31.8 |
| 14 | 3.99 | 68.7 |
| 15 | 1.3, 1.92 | 41.78 |
| 16 | 3.66 | 84.38 |
| 16-OCH₃ | 3.1 | 56.6 |
| 17 | | 139.38 |
| 17-CH₃ | 1.66 | 11.14 |
| 18 | 6.12 | 129.16 |
| 19 | 6.45 | 128.58 |
| 20 | 6.28 | 134.09 |
| 21 | 6.16 | 132.09 |
| 22 | 5.48 | 140.56 |
| 23 | 2.3 | 36.65 |
| 23-CH₃ | 1.02 | 22.1 |
| 24 | 1.12, 1.43 | 40.98 |
| 25 | 2.42 | 42.73 |
| 25-CH₃ | 0.86 | 13.55 |
| 26 | | 212.54 |
| 27 | 4.07 | 85.99 |
| 27-OCH₃ | 3.24 | 58.4 |
| 28 | 4.16 | 77.76 |
| 29 | | 137.45 |
| 29-CH₃ | 1.75 | 14.93 |
| 30 | 5.24 | 126.39 |
| 31 | 3.26 | 46.97 |
| 31-CH₃ | 0.95 | 16.78 |
| 32 | | 209.5 |
| 33 | 2.61, 2.7 | 46.09 |
| 34 | 5.19 | 72.74 |
| 35 | 1.5, 1.54 | 33.38 |
| 36 | 1.16, 1.24 | 32.75 |
| 37 | 1.25 | 36.96 |
| 38 | 0.7, 2.06 | 36.49 |
| 39 | 2.85 | 85.78 |
| 39-OCH₃ | 3.34 | 57.46 |
| 40 | 3.23 | 75.04 |
| 41 | 1.2, 1.84 | 33.35 |
| 42 | 0.9, 1.61 | 31.71 |

TABLE 4
¹H and ¹³C NMR signals assignment of 35-desmethy-27-demethoxy-rapamycin
(RAP35/27A) (major isomer, rotamer of trans form at the amide bond, in acetonitrile-d3)

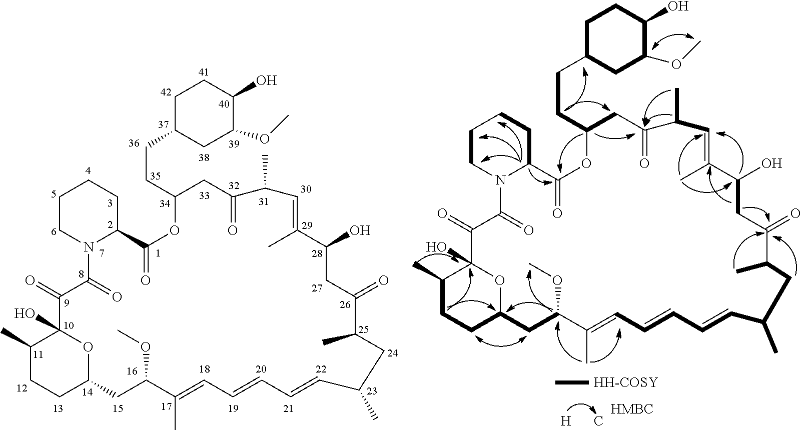

| Position | ¹H shift (ppm) | ¹³C shift (ppm) |
|---|---|---|
| 1 | | 170.77 |
| 2 | 5.03 | 52.78 |
| 3 | 1.67, 2.2 | 27.42 |
| 4 | 1.38, 1.7 | 21.63 |
| 5 | 1.38, 1.62 | 25.61 |
| 6 | 3.12, 3.47 | 45.38 |
| 8 | | 168.02 |
| 9 | | 198.16 |
| 10 | | 100.02 |
| 11 | 2.04 | 35.76 |
| 11-CH₃ | 0.84 | 16.47 |
| 12 | 1.58, 1.64 | 27.85 |
| 13 | 1.27, 1.86 | 31.37 |
| 14 | 4.01 | 68.61 |
| 15 | 1.35, 1.9 | 41.14 |
| 16 | 3.6 | 84.32 |
| 16-OCH₃ | 3.08 | 56.16 |
| 17 | | 138.36 |
| 17-CH₃ | 1.65 | 10.85 |
| 18 | 6.04 | 129.46 |
| 19 | 6.4 | 128.06 |
| 20 | 6.17 | 134.13 |
| 21 | 6.11 | 130.73 |
| 22 | 5.32 | 141.83 |
| 23 | 2.2 | 38.77 |
| 23-CH₃ | 1.00 | 22.21 |
| 24 | 1.29, 1.72 | 40.98 |
| 25 | 2.48 | 46.37 |
| 25-CH₃ | 0.97 | 17.67 |
| 26 | | 215.13 |
| 27 | 2.45, 2.61 | 49.04 |
| 28 | 4.25 | 71.95 |
| 29 | | 141.27 |
| 29-CH₃ | 1.57 | 13.96 |
| 30 | 5.23 | 124.32 |
| 31 | 3.3 | 46.8 |
| 31-CH₃ | 1.01 | 16.24 |
| 32 | | 209.04 |
| 33 | 2.66, 2.71 | 46.16 |
| 34 | 5.25 | 71.95 |
| 35 | 1.5, 1.54 | 32.86 |
| 36 | 1.17, 1.23 | 32.56 |
| 37 | 1.26 | 36.69 |
| 38 | 0.7, 2.05 | 36.2 |
| 39 | 2.85 | 85.49 |
| 39-OCH₃ | 3.34 | 57.16 |
| 40 | 3.23 | 74.73 |
| 41 | 1.2, 1.84 | 33.05 |
| 42 | 0.9, 1.6 | 31.64 |

SPR assays of interaction kinetics of GST-FKBP•Rapamycin and GST-FKBP•Rapamycin analogs for FRB

### Cloning, Overexpression and Purification of FRB

FRB was overexpressed and purified according to the method described previously with modifications. To construct the expression plasmid, the primers:

frb_pGEX_4T_3_F
(SEQ ID NO: 22)
5′-GACGAATCTCAAAGCAGTAGAATTCCCGGGTCGACTCGAG-3′
and
frb_pGEX_4T_3_R
(SEQ ID NO: 23)
5′-ATGGCCACTCGGATCAGCTCGGATCCACGCGGAACCAGAT-3′

were used to amplify the backbone fragment from pGEX-4T-3. The resulting 5-kb fragment was assembled with the synthesized gene fragment, frb, to afford pGEX-FRB. After validating the sequence of its insertion, pGEX-FRB was transformed into E. coli BL21(DE3) to overexpress GST tagged FRB. A single colony was picked to grow overnight at 37° C. in a 25 mL LB medium containing carbenicillin. The overnight culture was then inoculated (1:100 v/v) into 800 mL of LB medium containing carbenicillin. The culture was grown at 37° C. until OD600 reached 0.6 and then cooled down on ice for 30 min. Isopropyl-β-D-thiogalactopyranoside (IPTG, 0.3 mM final concentration) was added in the culture to induce the recombinant protein overexpression for 20 h at 18° C., and the cells were harvested by centrifugation (5,000 g, 10 min, 4° C.). To purify FRB, the harvested cells were resuspended in 30 mL PBS buffer (GE Healthcare, IL), and lysed by sonication on ice. After configuration (20,000 g, 30 min, 4° C.) to remove cellular debris, the supernatant was transferred to a new falcon tube. 1 mL Pierce™ Glutathione Agarose (Thermo Fisher Scientific, MA) was washed by PBS buffer and added into the supernatant and mixed for 1 hour at 4° C. The mixture was then loaded onto a Bio-Rad disposable column, and the resin was washed with 40 mL PBS buffer. After eluting the protein with 50 mM Tris-HCl, 10 mM glutathione (Thermo Fisher Scientific, MA), pH 7.5, buffer exchange was conducted using PD-10 Desalting Column (GE Healthcare, IL) to cleavage buffer (50 mM Tris-HCl, 100 mM NaCl, 10 mM CaCl₂, pH 8.0). The purity of the GST tagged protein was checked by SDS-PAGE (on 8-16% Mini-PROTEAN® TGX™ Precast Gels, Bio-Rad, CA), and its concentration was determined by the absorbance at 280 nm. 5 mg of the purified GST tagged FRB was taken and diluted using cleavage buffer to 2.5 mL, then 250 μL thrombin agarose, from Thrombin CleanCleave™ Kit (MilliporeSigma, MA) and prewashed by cleavage buffer, was added. The mixture was incubated at 4° C. for 16 hours with gentle agitation to keep beads suspended, and then loaded onto a Bio-Rad disposable column. The flow-through was collected, and 0.5 ml PBS washed glutathione agarose was added. The mixture was incubated at 4° C. for 2 hours with gentle agitation, and then loaded onto a Biorad disposable column. The flow-through was collected and subject to SEC using a Superdex 75 Increase 5/150 GL column (GE Healthcare, IL), run with PBS buffer at a flow rate of 0.4 mL/min. The fractions containing FRB (checked by SDS-PAGE) were pooled and concentrated using a 3 kDa MWCO amicon Ultra filter (MilliporeSigma, MA) to 0.1 mg/mL. The protein solution was aliquoted, flash-frozen in liquid nitrogen, and stored at −80° C.

Sequence of synthesized gene block frb:

(SEQ ID NO: 24)
ATCTGGTTCCGCGTGGATCCGAGCTGATCCGAGTGGCCATCCTCTGGCA
TGAGATGTGGCATGAAGGCCTGGAAGAGGCATCTCGTTTGTACTTTGGG
GAAAGGAACGTGAAAGGCATGTTTGAGGTGCTGGAGCCCTTGCATGCTA
TGATGGAACGGGGCCCCCAGACTCTGAAGGAAACATCCTTTAATCAGGC
CTATGGTCGAGATTTAATGGAGGCCCAAGAGTGGTGCAGGAAGTACATG
AAATCAGGGAATGTCAAGGACCTCACCCAAGCCTGGGACCTCTATTATC
ATGTGTTCCGACGAATCTCAAAGCAGTAGAATTCCCGGGTCGACTCGAG

All buffers were filtered through 0.2 μm filter units, and the assays were performed at 25° C. To immobilize anti-GST antibody on two flow cells of a Series S CM5 chip (GE Healthcare, IL), GST Capture kit (GE healthcare, IL), amine coupling reagents and general running buffer, PBS buffer (GE Healthcare, IL) supplemented with 0.002%, were used. The flow rate was set at 10 μL/min. The chip's surface was activated for 7 min using a 1:1 ratio of 0.4 M 1-ethyl-3-(3-dimethylaminopropyl) carbodiimide hydrochloride (EDC) and 0.1 M N-hydroxysuccinimide (NHS). Anti-GST antibody was diluted to 30 μg/ml using the immobilization buffer and injected over the surface for 5 min. Excess activated groups on the surface were blocked using 1 M ethanolamine hydrochloride-NaOH, pH 8.5, for 7 min, and high affinity sites of the immobilized antibody were blocked using 3 cycles of a 3-minute injection of recombinant GST at 5 μg/ml in the general running buffer followed by a 2-minute injection of the regeneration solution (10 mM glycine-HCl, pH 2.1). Approximately 9000 RU of the antibody was immobilized on each flow cell.

To perform the single-cycle kinetics assays, 50 μM rapamycin and rapamycin analogs in ethanol were added into the general running buffer (1:1000 v/v) to make the running buffers, respectively. All the reagents were diluted in the corresponding running buffer in each assay. Three times of prime procedure were conducted after changing the running buffer. The single-cycle kinetics analysis method begins with two startup cycles, followed by the sample cycles for blank and FRB. In the startup cycle applied to both flow cells, the running buffer was injected at a flow rate of 50 μL/min for 3 min. A 2-minute injection of the regeneration solution was then conducted at a flow rate of 20 μL/min, followed by a 30-s stabilization period. In the assay cycle, 0.2 mg/mL GST-FKBP1A (Creative BioMart Inc., NY, buffer exchanged to the general running buffer prior to assays) was injected to the active flow cell at a flow rate of 20 μL/min for 30 s. 0.2 mg/mL recombinant GST (Thermo Fisher Scientific, MA) was injected to the reference flow cell at a flow rate of 20 μL/min for 30 s. Following the GST tagged proteins immobilization, samples were injected to both flow cells using a single cycle kinetics method with five FRB concentrations at a flow rate of 50 μL/min. The contact time was set at 60 s, and the dissociation time was set at 120 s. In the blank cycle, the running buffer (FRB concentration=0) was used as samples. In the FRB cycle, the FRB sample concentrations were 8 nM, 16 nM, 32 nM, 64 nM, and 128 nM. After sample injections, a 2-minute injection of the regeneration solution was conducted at a flow rate of 20 μL/min for both flow cells, followed by a 30-s stabilization period.

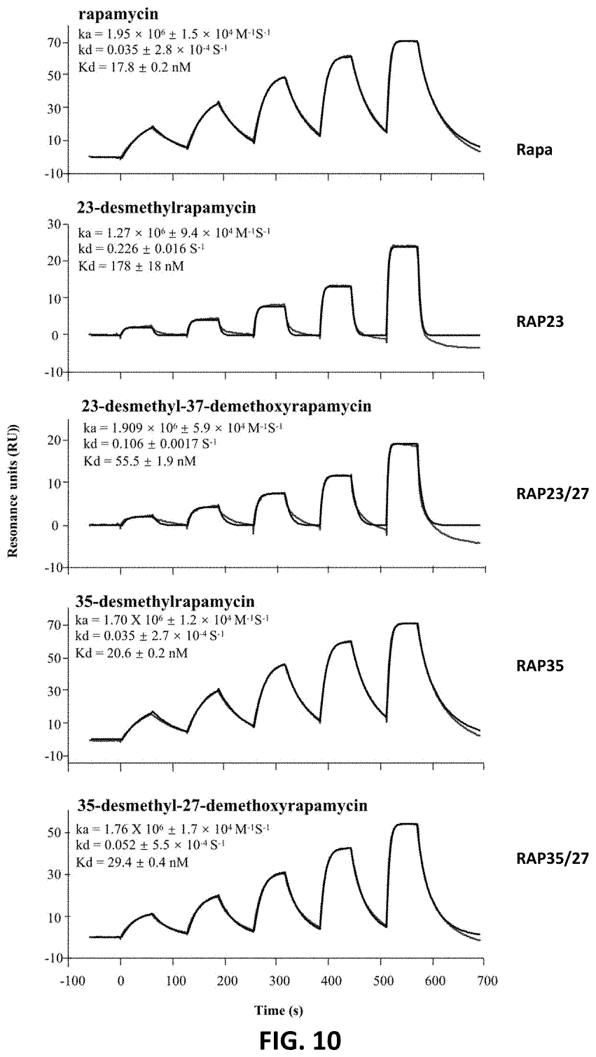

The data analysis was performed using Biacore T200 Evaluation Software (v3.1). The response curves of the active flow cell minus the reference cell were used. The blank subtracted FRB titration curves were fit to the 1:1 binding model using the surface bound kinetics evaluation. The results show that all the rapamycin analogs were able to mediate the formation of FKBP-rapamycin analog-FRB complex, with varying potency (FIG. 10).

Flow Cytometry (Phosphor-Flow) to Screen Compounds for their Ability to Inhibit mTOR in Activated T Cells

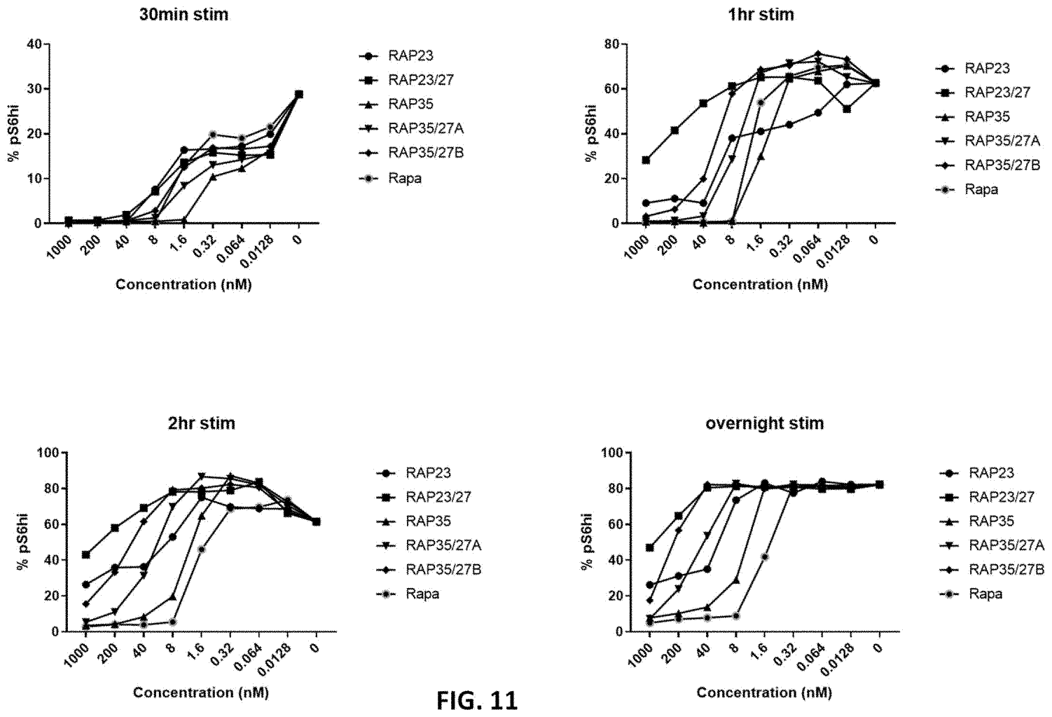

Whole splenocyte from OT1 B6 mice was extracted. The T cells were activated with soluble anti-CD3 (3 μg/mL), anti-CD28 (2 μg/mL), and IgG (1.5 μg/mL), along with rapamycin, rapamycin analogs, and DMSO, respectively. Cells were activated with compounds for 30 mins, 1 hour, 2 hours, and overnight. For each time point, p-S6, a downstream of mTORC1 pathway, was gated on low and high based on the DMSO and no stimulation control. FIG. 11 shows the percentage of p-S6^{hi} cells after 30 mins, 1 hour, 2 hours, and overnight of activation. All rapamycin analogs can inhibit mTOR activity in activated T cells, with varying potency.

### Anti-Proliferation Assay

Assay of CD8 CVT-Labelled Proliferation with Rapamycin Analogs

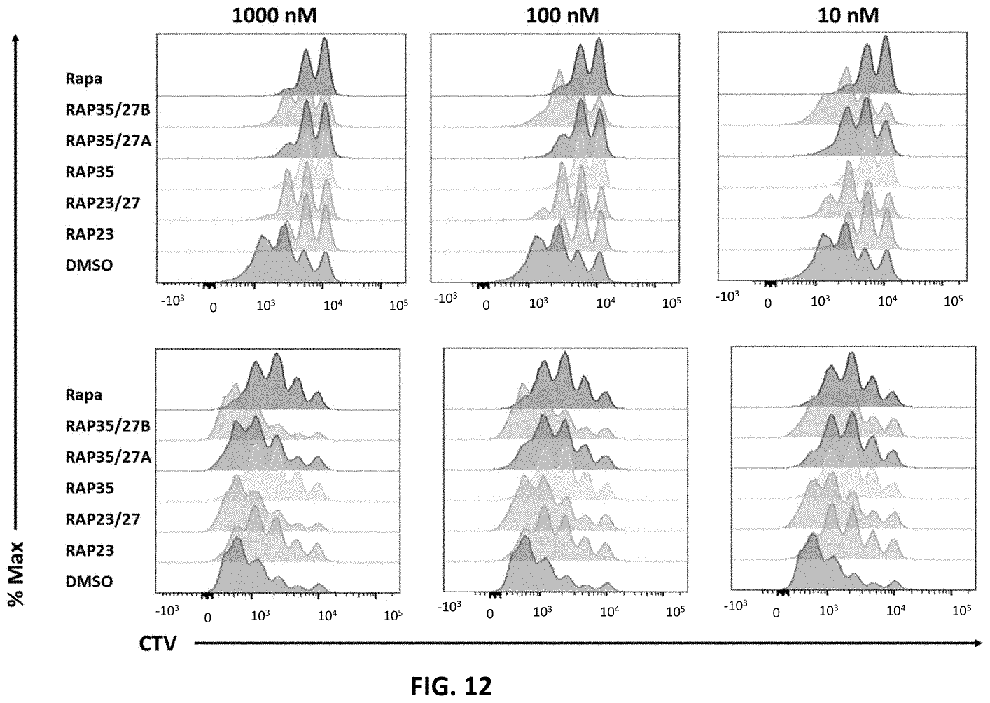

CD8 cells were isolated from p14 transgenic mice, then labelled with CTV. The labelled cells then were activated with plate-bound anti-CD3 (5 μg/mL) and soluble anti-CD28 (2 μg/mL), along with rapamycin, rapamycin analog, or DMSO. After activation of 48 hours and 72 hours, the cells were collected to assay the CVT labelling signal using flow cytometry. All rapamycin analogs decrease proliferation in CD8 cells, with varying potency (FIG. 12).

Regulatory T Cell (Treg) Generation with Rapamycin Analogs

CD4 cells were isolated from OT2 mice and activated with plate-bound anti-CD3 (5 μg/mL), soluble anti-CD28 (2 μg/mL), soluble IL-2 (1 ng/mL) and TGFb (1 ng/mL). Cells were given 1000 nM of each rapamycin analog or rapamycin as control.

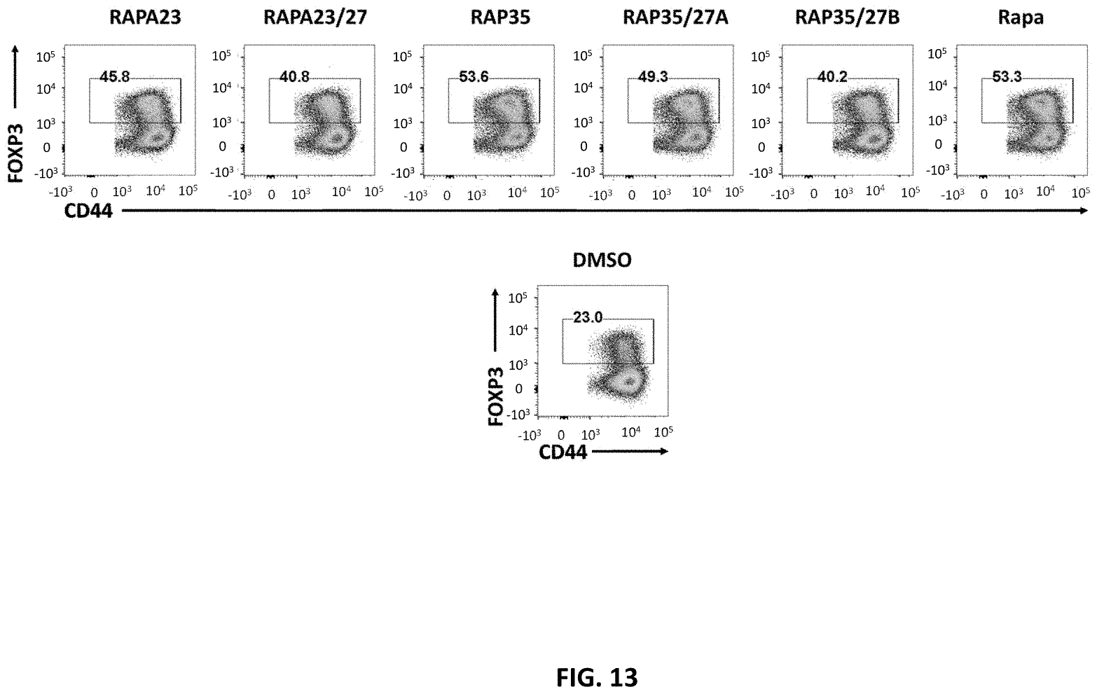

The rapamycin analogs show increase in Treg generation in the presence of IL-2 and TGFb (FIG. 13).

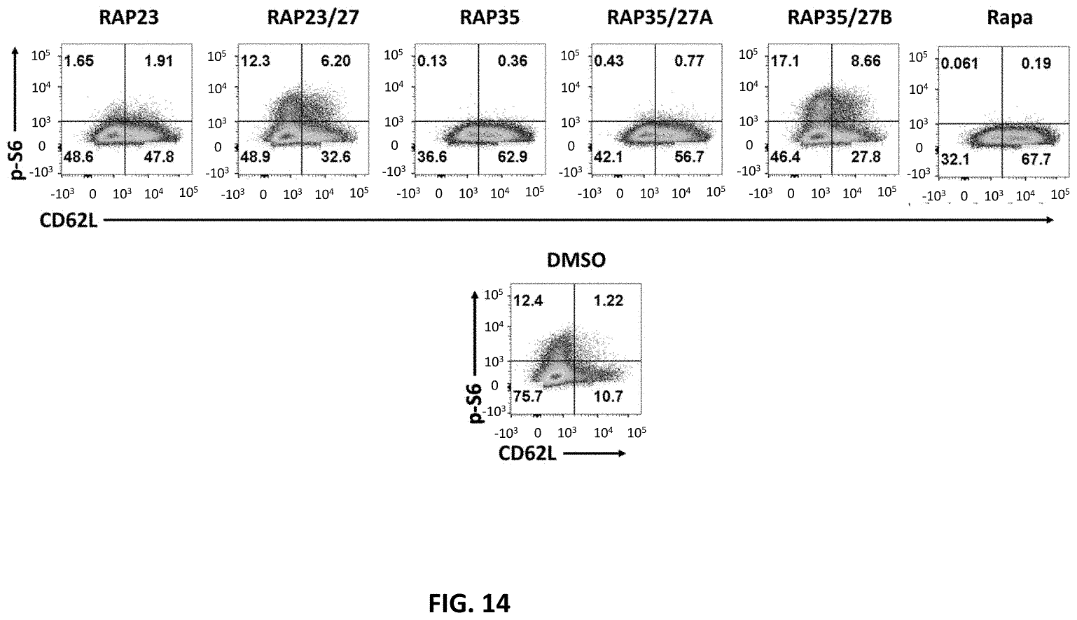

The rapamycin analogs increase CD62L+ Tregs, central Treg with memory phenotype (FIG. 14).

### T_{reg} Assays for RAP35

Culture of conventional T cells with RAP35 saw promising anti-inflammatory effects through the readouts from Cell Proliferation Dye and phenotypic markers such as increased CCR7 and decreased CD45RO expression, as well as the level of phosphorylation of 4EBP1 and S6, indicating the mTOR pathway was being targeted and was the mechanism of action for 100 nM RAP35. MitoProbe analysis for mitochondrial health also showed reduced mitochondrial membrane potential for RAP35 compared to stimulated CD3 T cells. Treatment of T_{reg} cells with expander beads alongside the therapeutics showed equivalent effects to the conventional T cells with similar readouts. 100 nM RAP35 was shown to reduce the frequency of T_{reg} cells, expression of CCR7, CXCR3 and CD45RO as well as phosphorylation of 4EBP1 and S6.

### General Materials and Methods (for Examples 3 and 4)

For the growth and sporulation of S. rapamycinicus on plate, ISP Medium No. 3 agar (HiMedia Laboratories Pvt. Ltd., India) was used. For the growth of S. rapamycinicus in liquid culture, Bacto Tryptic Soy Broth (BD, NJ) with 10 g/L glucose, pH 6.0, was used. For the growth of E. coli, pre-poured Luria-Bertani (LB) agar plates (Teknova Inc, CA) and LB broth (miller, Merck KGaA, Darmstadt, Germany) were used. For the conjugation between E. coli and S. rapamycinicus, pre-poured Mannitol Soy (MS) agar plate (with 10 mM magnesium chloride) was used. For the growth of Saccharomyces cerevisiae, YPD agar plate (Teknova Inc, CA) was used. Antibiotics were added in these medias when needed as following concentrations: carbenicillin, 100 mg/L for E. coli; chloramphenicol, 25 mg/L for E. coli; kanamycin, 50 ml/L for E. coli; apramycin, 50 mg/L for E. coli, 20 mg/L for S. rapamycinicus. Chemicals were purchased from Millipore Sigma (Merck KgaA, Darmstadt, Germany) unless otherwise indicated.

Primers were purchased from Integrated DNA Technologies (IDT, Coralville, Iowa). Polymerase chain reaction (PCR) amplifications were performed on an Applied Biosystems Veriti Thermal Cycler (Thermo Fisher Scientific, MA) using PrimeSTAR Max DNA Polymerase (Takara Bio Inc., Japan). Gene fragments encoding the AT8 of rapamycin PKS and FRB were synthesized by Integrated DNA Technologies (IDT, Coralville, Iowa). Plasmids were assembled using NEBuilder HiFi DNA Assembly Master Mix (New England Biolabs, MA) unless otherwise indicated. Plasmid isolation was carried out using QIAprep Spin Miniprep Kit (Qiagen, Germany). DNA purification was carried out using Sanger sequencing service was supplied by Genewiz, San Francisco, Calif.

High-performance liquid chromatography analysis (HPLC) was performed on an Agilent 1260 HPLC system (Agilent Technologies Inc., CA) equipped with a diode array detector (DAD). Semi-preparation of small molecules was performed on the same system. All ions tandem mass spectrometry (MS/MS) analysis was performed on an Agilent 6520 Accurate-Mass Q-TOF LC/MS System (Agilent Technologies Inc., CA) with collision energy of 40V. HPLC-DAD data were analyzed using ChemStation, and LC-MS/MS data were analyzed using MassHunter (Agilent Technologies Inc., CA). Nuclear magnetic resonance (NMR) data were recorded at Central California 900 MHz NMR Facility of QB3-Berkeley and analyzed using MestReNova. Surface plasmon resonance (SPR) was performed on a Biacore T100 with T200 sensitivity enhancement (GE Healthcare, IL). Size exclusion chromatography (SEC) was performed on an ÄKTA Explorer fast protein liquid chromatography (FPLC) system (GE Healthcare, IL).

While preferred embodiments of the present disclosure have been shown and described herein, it may be obvious to those skilled in the art that such embodiments are provided by way of example only. Numerous variations, changes, and substitutions may now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. A compound, or a pharmaceutically-acceptable salt or solvate thereof, having a structure represented by a structure of Formula (I): 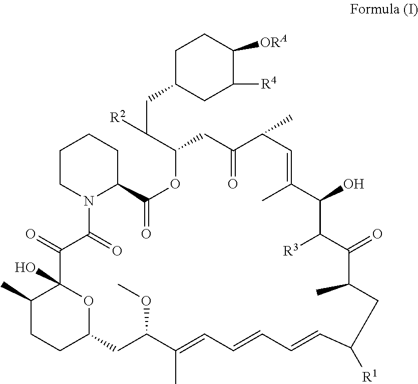
wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R¹ is hydrogen or CH₃;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R⁴ is —OCH₃ or 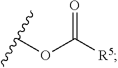
R⁵ is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl;
wherein when R¹ is hydrogen, at least one of (i) R² is not CH₃, (ii) R³ is not —OCH₃, or
(iii) R⁴ is not —OCH₃;
and
wherein when R¹ is CH₃, at least one of (i) R² is not CH₃, (ii) R³ is not hydrogen, —CH₃ or —OCH₃, or (iii) R⁴ is not —OCH₃.

2. The compound of claim 1, wherein R⁴ is —OCH₃.

3. The compound of claim 1, wherein R¹ is hydrogen or CH₃.

4. The compound of claim 2, wherein R¹ is hydrogen.

5. The compound of claim 1 wherein R² is CH₃.

6. The compound of claim 1, wherein:
R¹ is hydrogen;
R² is CH₃;
R³ is hydrogen; and
R⁴ is —OCH₃.

7. The compound of claim 1, wherein R¹ is CH₃.

8. The compound of claim 1, wherein R² is hydrogen or CH₃.

9. The compound of claim 8, wherein R² is hydrogen.

10. The compound of claim 1, wherein:
R¹ is CH₃;
R² is hydrogen;
R³ is —OCH₃; and
R⁴ is —OCH₃.

11. The compound of claim 1, wherein:
R¹ is CH₃;
R² is hydrogen;
R³ is hydrogen; and
R⁴ is —OCH₃.

12. The compound of claim 1, wherein R¹ is hydrogen, R² is CH₃, and R⁴ is —OCH₃.

13. The compound of claim 1, wherein R¹ is an optionally substituted alkyl.

14. The compound of claim 1, wherein R² is an optionally substituted alkyl.

15. The compound of claim 1, wherein R³ is an optionally substituted alkoxy.

16. The compound of claim 1, wherein R⁴ is an optionally substituted alkoxy.

17. A compound, or a pharmaceutically-acceptable salt or solvate thereof, having a structure represented by a structure of Formula (I-A): 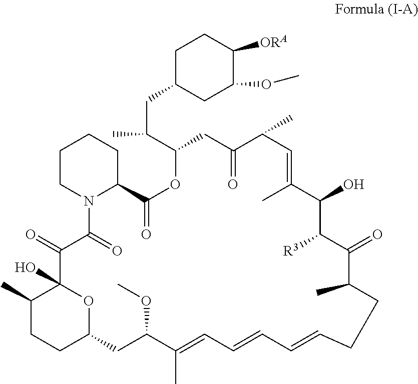
wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the alkyl, cycloalkyl, heterocycloalkyl, aryl, and heteroaryl are each independently optionally substituted with one or more halogen, —OH, —NH₂, or C₁-C₆ alkyl; and
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl.

18. The compound of claim 17, wherein R³ is hydrogen.

19. A compound, or a pharmaceutically-acceptable salt or solvate thereof, having a structure represented by a structure of Formula (I-B): 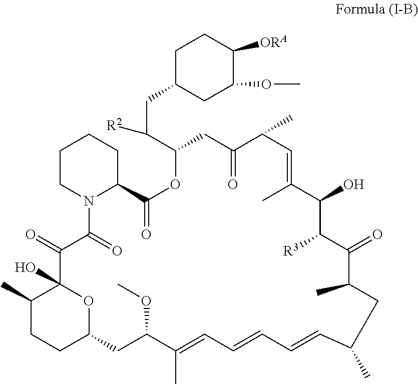
wherein,
R^{A} is hydrogen or —C(═O)R^{B}, wherein R^{B} is C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R² is hydrogen, halogen, —CN, —OR²¹, —SR²¹, —S(═O)R²², —S(═O)₂R²², —NO₂, —NR²³R²⁴, —NR²¹S(═O)₂R²², —S(═O)₂NR²³R²⁴, —C(═O)R²², —OC(═O)R²², —C₁-C₆—C(═O)R²⁰, —C(═O)C(═O)R²², —C(═O)OR²¹, —C(═O)NR²¹OR²¹, —OC(═O)OR²¹, —C(═O)NR²³R²⁴, —OC(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
R³ is hydrogen, halogen, —OCH₃, —CN, —SR²¹, —S(═O)R²², —S(═O)₂R²², —NO₂, —NR²³R²⁴, —NR²¹S(═O)₂R²², —S(═O)₂NR²³R²⁴, —C(═O)R²², —C(═O)C(═O)R²², —C₁-C₆—C(═O)R²⁰, —C(═O)OR²¹, —C(═O)NR²¹OR²¹, —C(═O)NR²³R²⁴, —NR²¹C(═O)NR²³R²⁴, —NR²¹S(═O)₂NR²³R²⁴, —NR²¹C(═O)R²², —NR²¹C(═O)OR²¹, C₁-C₆ alkyl, C₂-C₆ alkenyl, cycloalkyl, heterocycloalkyl, aryl, or heteroaryl; wherein the C₁-C₆ alkyl, C₂-C₆ alkenyl, cycloalkyl, heterocycloalkyl, aryl, and heteroaryl are each independently optionally substituted with one or more R²⁰;
each R²⁰ is independently halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R²¹ is independently hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one R^{1a};
R²² is hydrogen, —CN, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1b};
R²³ and R²⁴ are each independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more R^{1c};
or R²³ and R²⁴ are taken together with the nitrogen atom to which they are attached to form a heterocycloalkyl optionally substituted with one or more R^{1d};
each R^{1a}, R^{1b}, R^{1c}, and R^{1d} are independently oxo, halogen, —CN, —OR^{a}, —S(═O)₂R^{b}, —NR^{c}R^{d}, —S(═O)₂NR^{c}R^{d}, —C(═O)R^{b}, —OC(═O)R^{b}, —C(═O)OR^{a}, —OC(═O)OR^{a}, —C(═O)NR^{c}R^{d}, —OC(═O)NR^{c}R^{d}, —NR^{a}C(═O)NR^{c}R^{d}, —NR^{a}C(═O)R^{b}, C₁-C₆ alkyl, C₁-C₆ haloalkyl, C₁-C₆ hydroxyalkyl, or phenyl;
each R^{a} is independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{b} is independently C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
each R^{c} and R^{d} are independently hydrogen, C₁-C₆ alkyl, cycloalkyl, heterocycloalkyl, aryl, or heteroaryl; wherein the C₁-C₆ alkyl, cycloalkyl, heterocycloalkyl, aryl, and heteroaryl are each independently optionally substituted with one or more halogen, —OH, —NH₂, or C₁-C₆ alkyl;
or R^{c} and R^{d} are taken together with the nitrogen atom to which they are attached to form a heterocycloalkyl optionally substituted with one or more halogen, —OH, —NH₂, or C₁-C₆ alkyl;
and
wherein when R² is CH₃, R³ is not hydrogen, —CH₃ or —OCH₃.

20. The compound of claim 19, wherein R² is hydrogen or CH₃.

21. The compound of claim 20, wherein R² is hydrogen.

22. The compound of claim 19, wherein R² is hydrogen and R³ is —OCH₃.

23. The compound of claim 19, wherein R² is hydrogen and R³ is hydrogen.

24. The compound of claim 19, wherein R² is an optionally substituted alkyl.

25. The compound of claim 19, wherein R³ is an optionally substituted alkoxy.

26. The compound of claim 1, wherein the compound of Formula (I), or pharmaceutically acceptable salt or solvate thereof, is selected from the group consisting of:

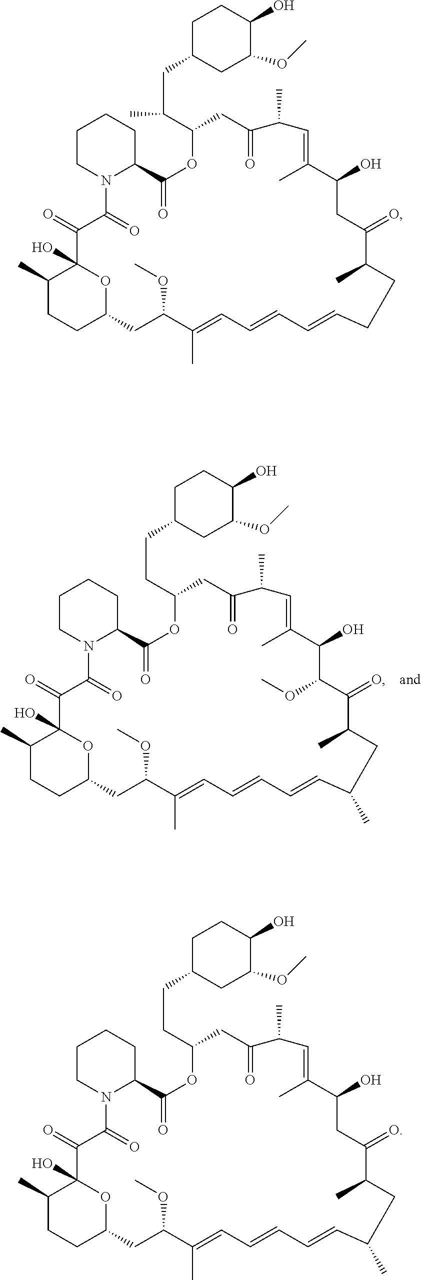

27. A pharmaceutical composition comprising the compound of claim 1 and at least one pharmaceutically-acceptable excipient.

28. The pharmaceutical composition of claim 27, wherein the pharmaceutical composition is in a unit dosage form.

29. A kit comprising the pharmaceutical composition of claim 27.

30. The kit of claim 29, further comprising a coronavirus vaccine.
